(19) United States
(12) Patent Application Publication
(10) Pub. No.: US 2024/0100031 A1
(43) Pub. Date: Mar. 28, 2024

(54) PHARMACEUTICAL COMPOSITION FOR PREVENTING OR TREATING VIRAL INFECTIONS CONTAINING PIPERLONGUMINE-BASED COMPOUND AS ACTIVE INGREDIENT

(71) Applicant: HUSCION CO., LTD., Seongnam-si, Gyeonggi-do (KR)

(72) Inventors: Seong Soo JOO, Yongin-si, Gyeonggi-do (KR); Nareum HA, Yongin-si, Gyeonggi-do (KR); Jungmin YU, Gwangju-si, Gyeonggi-do (KR); Youngsoo GYOUNG, Gwangju-si, Gyeonggi-do (KR); Jonggab JUN, Chuncheon-si, Gangwon-do (KR); Heonse JEONG, Seoul (KR); Sukil JANG, Gangneung-si, Gangwon-do (KR)

(73) Assignee: HUSCION CO., LTD., Seongnam-si, Gyeonggi-do (KR)

(21) Appl. No.: 18/269,398

(22) PCT Filed: Dec. 27, 2021
(86) PCT No.: PCT/KR2021/019942

§ 371 Date: Jun. 23, 2023

(30) Foreign Application Priority Data
Dec. 29, 2020 (KR) 10-2020-0185404
Dec. 23, 2021 (KR) 10-2021-0185813

(51) Int. Cl. A61K 31/4412; A61K 31/235; A61K 31/4015; A61K 31/4025; A61K 31/4439; A61K 31/495; A61P 31/14

(52) U.S. Cl. CPC A61K 31/4412; A61K 31/235; A61K 31/4015; A61K 31/4025; A61K 31/4439; A61K 31/495; A61P 31/14

(57) ABSTRACT

The present disclosure relates to a pharmaceutical composition for preventing or treating viral infections, containing a piperlongumine-based compound as an active ingredient. Specifically, the piperlongumine-based compound according to the present disclosure inhibits SARS-CoV-2 infection, and thus can be useful for treating viral infections, particularly RNA viral infections.

### TECHNICAL FIELD

The present disclosure relates to a pharmaceutical composition for preventing or treating viral infections, containing a piperlongumine-based compound as an active ingredient.

### BACKGROUND ART

Viruses are living organisms that have an average size of 0.1 μm and contain only nucleic acids and a small number of proteins necessary for their survival, and thus depend on a host such as humans to survive. Since viruses have very different ways of invading and surviving the host according to the types thereof, reactions in a host during infection also appear to be very diverse. Since it is known that common viral infections repeat the process of penetration into host cells, synthesis and assembly of new viruses, and cell releasing, currently, materials that suppress viral penetration, intracellular proliferation, or the like are mainly being developed as antiviral agents.

Viruses have one or two strands of genetic material, and these genetic materials are wrapped in a coat composed of proteins, and thus act as a protective barrier from the outside in some cases. Accordingly, the host's immune system recognizes the surface protein of the virus as an antigen to protect the host from the virus, but when mutation occurs according to the structure and characteristics of the virus, the mutant may not be recognized as an antigen in some cases. In such cases, a severe pandemic may occur locally or internationally.

Immunotherapy using vaccines is the mainstream of treatment for viral infections, and various chemically synthesized drugs and biopharmaceuticals such as interferon have also been used. However, for RNA viruses such as a novel coronavirus (SARS-CoV-2), which is currently causing a worldwide pandemic, there is a limitation of immunotherapy using vaccines due to the infestation of various mutants. Meanwhile, for the treatment of infectious diseases caused by RNA viruses, it is possible to use inhibitors which suppress cell membrane fusion between viruses and host cells, reverse transcription polymerase inhibitors which convert RNA into DNA, proteolytic enzyme inhibitors which suppress the cleavage of viral polyproteins, and the like. However, most of the drugs currently used as therapeutic agents for viral infections cannot completely suppress the production of viruses, resulting in the generation of drug-resistant viruses, or side effects such as immune system disorders and toxicity.

In this regard, Korean Patent No. 10-2337933 relates to a 1,2,4-oxadiazole derivative compound and an antiviral agent containing the same as an active ingredient, and confirmed that the 1,2,4-oxadiazole derivative compound exhibits antiviral activity without cytotoxicity.

### DISCLOSURE

### Technical Problem

An object of the present disclosure is to provide a use of a piperlongumine-based compound for treating viral infections.

### Technical Solution

To achieve the object, the present disclosure provides a pharmaceutical composition for preventing or treating viral infections, containing a compound represented by the following Chemical Formula 1, and a pharmaceutically acceptable salt or solvate thereof as an active ingredient:

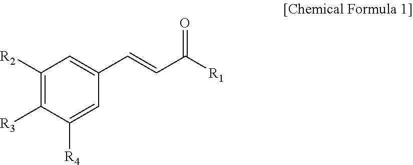

in Chemical Formula 1,

R₂ to R₄ are the same as or different from each other, and are each independently selected from the group consisting of hydrogen, deuterium, a halogen, a nitro group, a hydroxyl group, an amino group, a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₁˜C₂₀ alkoxy group, a C₁˜C₂₀ ketone group, a C₁˜C₂o ester group, a C₆˜C₂₀ aryl group, a heteroaryl group having 5 to 20 nuclear atoms, and a C₆˜C₂₀ aryloxy group, or these may be bonded to an adjacent group to form a C₆˜C₂o aryl or a heteroaryl ring having 5 to 20 nuclear atoms, provided that the case where R₂ to R₄ are the same is excluded,

R₁ is a substituent selected from the following structural formulae,

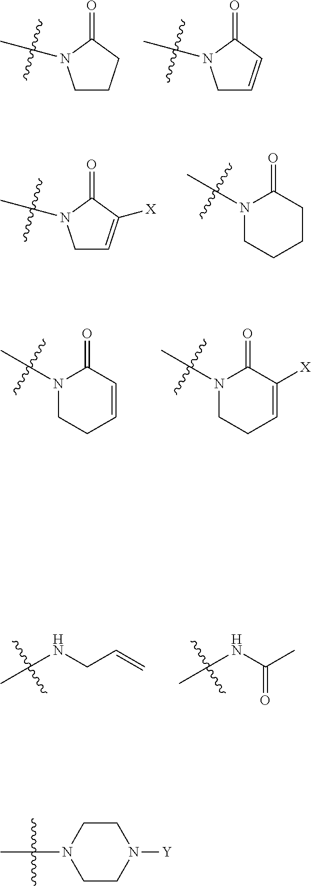

in the formulae,

the wavy part means a part that is bonded to Chemical Formula 1,

X is a halogen element selected from the group consisting of F, Cl, Br and I,

Y is a C₁˜C₁₀ alkyl group,

the alkyl group, the ketone group, the ester group, the aryl group, and the heteroaryl group of R₂˜R₄ may be each independently substituted with one or more substituents selected from the group consisting of deuterium, a halogen, a cyano group, a nitro group, a C₁˜C₄₀ alkyl group, a C₂˜C₄₀ alkenyl group, a C₂˜C₄₀ alkynyl group, a C₆˜C₄₀ aryl group, a heteroaryl group having 5 to 40 nuclear atoms, a C₆˜C₄₀ aryloxy group, a C₁˜C₄₀ alkyloxy group, a C₆˜C₄₀ arylamine group, a C₃˜C₄₀ cycloalkyl group, a heterocycloalkyl group having 3 to 40 nuclear atoms, a C₁˜C 40 alkylsilyl group, a C₁˜C₄₀ alkylboron group, a C₆˜C₄₀ arylboron group, a C₆˜C₄₀ arylphosphine group, a C₆˜C₄₀ arylphosphine oxide group and a C₆˜C₄₀ arylsilyl group, and in this case, when there is a plurality of substituents, the substituents may be the same as or different from each other.

Further, the present disclosure provides a health functional food for preventing or ameliorating viral infections, containing the above-described compound represented by Chemical Formula 1, and a pharmaceutically acceptable salt or solvate thereof as an active ingredient.

In addition, the present disclosure provides a method for preventing, ameliorating or treating viral infections, the method including administering the above-described compound represented by Chemical Formula 1, and a pharmaceutically acceptable salt or solvate thereof to an individual.

Furthermore, the present disclosure provides a use of the above-described compound represented by Chemical Formula 1, and a pharmaceutically acceptable salt or solvate thereof for use in the preparation of a drug for preventing, ameliorating, or treating viral infections.

### Advantageous Effects

The piperlongumine-based compound according to the present disclosure inhibits SARS-CoV-2 infection, and thus can be useful for treating viral infections, particularly RNA viral infections.

### DESCRIPTION OF DRAWINGS

FIG. 1 is a set of graphs showing the results of confirming the viral infection inhibitory activity of the compound according to the present disclosure using a pseudovirus of a novel coronavirus.

### BEST MODE

Hereinafter, the present disclosure will be described in detail.

The present disclosure provides a pharmaceutical composition for preventing or treating viral infections, containing a compound represented by the following Chemical Formula 1, and a pharmaceutically acceptable salt or solvate thereof as an active ingredient:

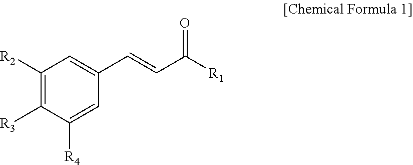

in Chemical Formula 1,

R₂ to R₄ are the same as or different from each other, and are each independently selected from the group consisting of hydrogen, deuterium, a halogen, a nitro group, a hydroxyl group, an amino group, a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₁˜C₂₀ alkoxy group, a C₁˜C₂₀ ketone group, a C₁˜C₂o ester group, a C₆˜C₂₀ aryl group, a heteroaryl group having 5 to 20 nuclear atoms, and a C₆˜C₂₀ aryloxy group, or these may be bonded to an adjacent group to form a C₆˜C₂o aryl or a heteroaryl ring having 5 to 20 nuclear atoms, provided that the case where R₂ to R₄ are the same is excluded,

R₁ is a substituent selected from the following structural formulae,

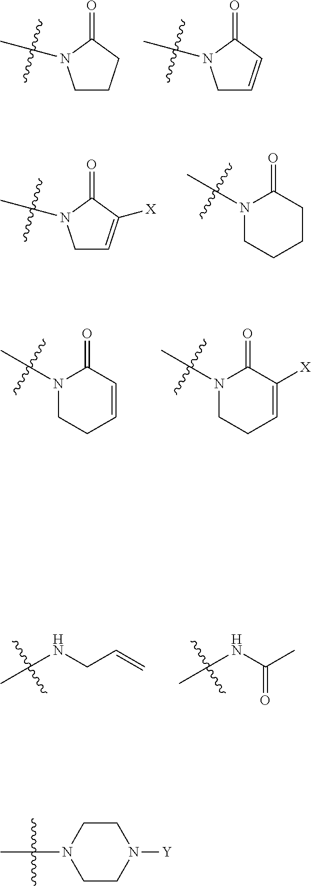

in the formulae,

the wavy part means a part that is bonded to Chemical Formula 1,

X is a halogen element selected from the group consisting of F, Cl, Br and I,

Y is a C₁˜C₁₀ alkyl group, and

the alkyl group, the ketone group, the ester group, the aryl group, and the heteroaryl group of R₂˜R₄ may be each independently substituted with one or more substituents selected from the group consisting of deuterium, a halogen, a cyano group, a nitro group, a C₁˜C₄₀ alkyl group, a C₂˜C₄₀ alkenyl group, a C₂˜C₄₀ alkynyl group, a C₆˜C₄₀ aryl group, a heteroaryl group having 5 to 40 nuclear atoms, a C₆˜C₄₀ aryloxy group, a C₁—C₄₀ alkyloxy group, a C₆˜C₄₀ arylamine group, a C₃˜C₄₀ cycloalkyl group, a heterocycloalkyl group having 3 to 40 nuclear atoms, a C₁—C 40 alkylsilyl group, a C₁˜C₄₀ alkylboron group, a C₆˜C₄₀ arylboron group, a C₆˜C₄₀ arylphosphine group, a C₆˜C₄₀ arylphosphine oxide group and a C₆˜C₄₀ arylsilyl group, and in this case, when there is a plurality of substituents, the substituents may be the same as or different from each other.

Specifically, R₁ of Chemical Formula 1 may have a substituent selected from the following structural formulae:

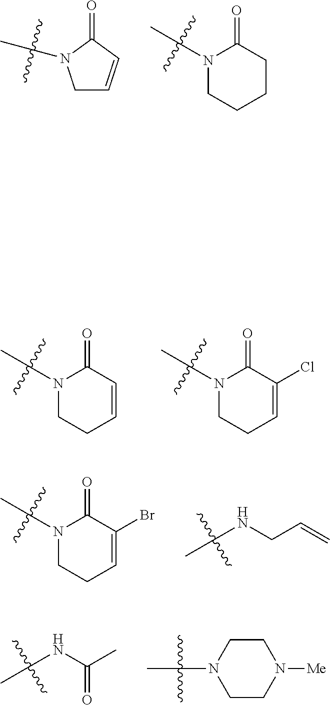

Further, R₂ to R₄ are the same as or different from each other, and are each independently selected from the group consisting of hydrogen, a nitro group, a hydroxyl group, an ester group including a C₁˜C 6 alkyl, and a C₁˜C 6 alkoxy group, or two adjacent substituents may be bonded to each other to form a C₆-C₁₀ aryl group, or a hetero ring having 5 to 10 nuclear atoms, including at least one of nitrogen and oxygen atoms.

In the present disclosure, the alkyl group, the ketone group, the ester group, the aryl group, and the heteroaryl group of R₂˜R₄ may be each independently substituted with one or more substituents selected from the group consisting of deuterium, a halogen, a cyano group, a nitro group, a Ci—C₄₀ alkyl group, a C₂˜C₄₀ alkenyl group, a C₂˜C₄₀ alkynyl group, a C₆˜C₄₀ aryl group, a heteroaryl group having 5 to 40 nuclear atoms, a C₆˜C₄₀ aryloxy group, a Ci—C₄₀ alkyloxy group, a C₆˜C₄₀ arylamine group, a C₃˜C₄₀ cycloalkyl group, a heterocycloalkyl group having 3 to 40 nuclear atoms, a C₁˜C₄₀ alkylsilyl group, a Ci—C₄₀ alkylboron group, a C₆˜C₄₀ arylboron group, a C₆˜C₄₀ arylphosphine group, a C₆˜C₄₀ arylphosphine oxide group and a C₆˜C₄o arylsilyl group, and in this case, when there is a plurality of substituents, the substituents may be the same as or different from each other.

More specifically, the compound of Chemical Formula 1 may be any one of the following Chemical Formulae 2 to 5 according to the type of R₂ to R₄:

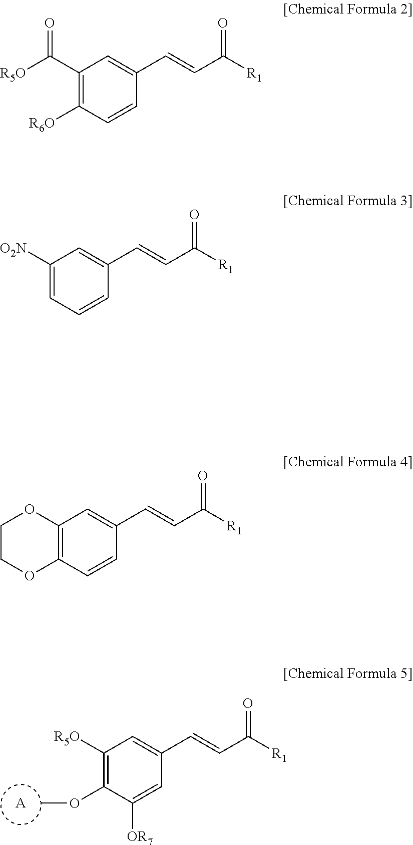

in Chemical Formulae 2 to 5,

R₁ is the same as that defined in Chemical Formula 1.

R₅ to R₇ are the same as or different from each other, and may be each independently selected from the group consisting of hydrogen, a hydroxyl group, a C₁˜C₆ alkyl group, a C₁˜C₆ alkoxy group, a C₁˜C₆ ketone group, and a C₁˜C₆ ester group.

Ring A may be a monocyclic or polycyclic hydrocarbon ring group having 20 or less carbon atoms, and may be specifically selected from the group consisting of a cycloalkyl ring, a heterocycloalkyl ring, an aryl ring and a heteroaryl ring. At least one carbon atom constituting such ring A may be substituted with one or more substituents selected from the group consisting of deuterium, a halogen, a cyano group, a nitro group, a C₁˜C₄₀ alkyl group, a C₆˜C₄₀ aryl group, and a heteroaryl group having 5 to 40 nuclear atoms.

As an example, the compound represented by Chemical Formula 1 of the present disclosure may be selected from the group consisting of the following exemplified compounds:

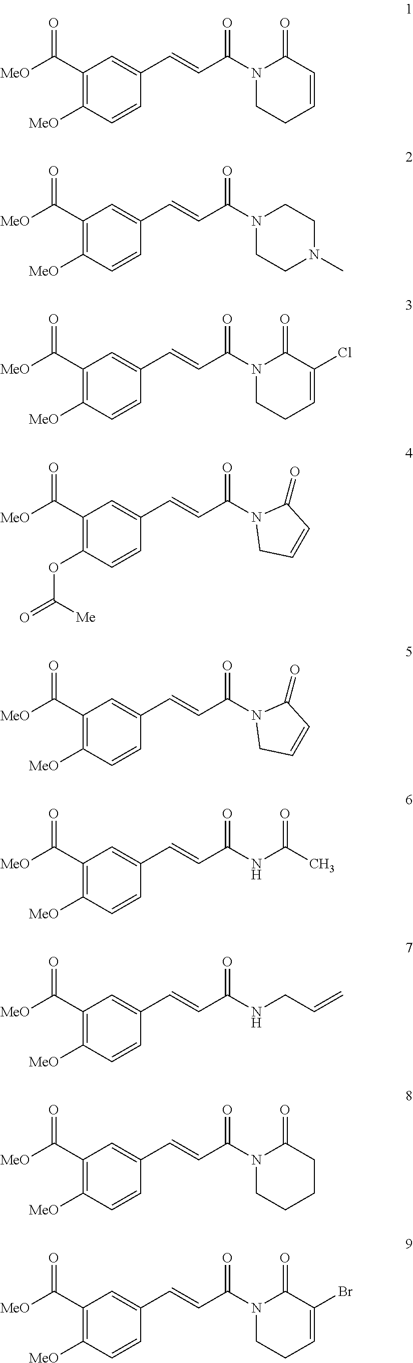

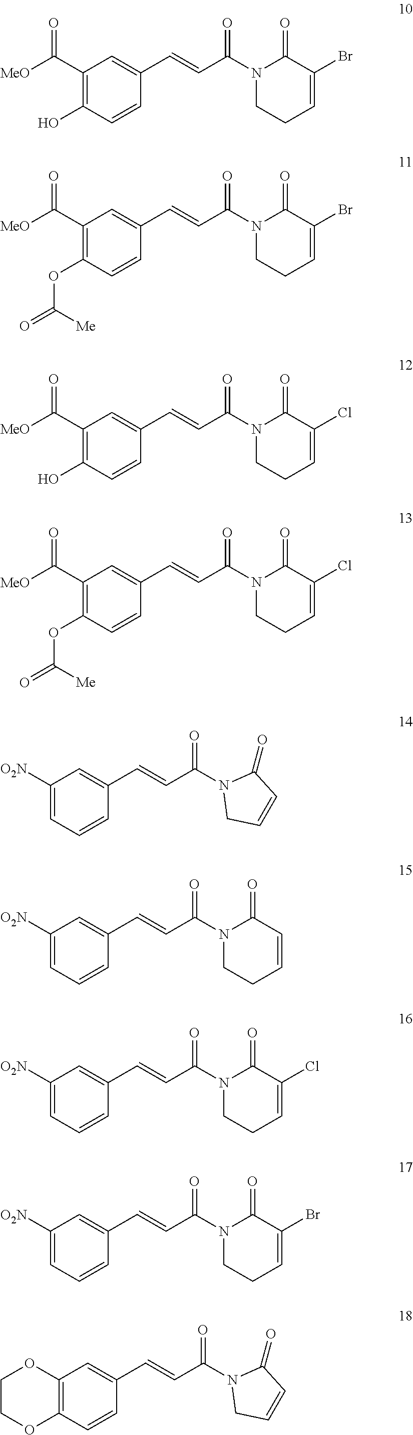

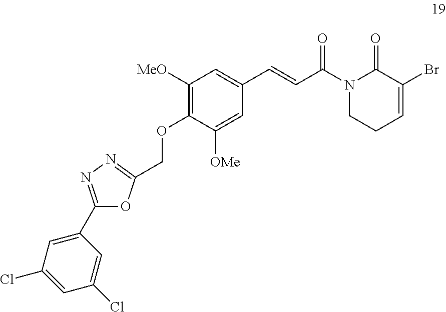 [SMILES: COc1cc(/C=C/C(=O)N2CCC=C(Br)C2=O)cc(OC)c1OCc1nnc(-c2cc(Cl)cc(Cl)c2)o1]

As used herein, the term “alkyl” refers to a monovalent substituent derived from a linear or branched saturated hydrocarbon having 1 to 40 carbon atoms.

Examples thereof may include methyl, ethyl, propyl, isobutyl, sec-butyl, pentyl, iso-amyl, hexyl, and the like.

As used herein, the term “alkenyl” refers to a monovalent substituent derived from a linear or branched unsaturated hydrocarbon having 2 to 40 carbon atoms and having one or more carbon-carbon double bonds. Specific examples thereof may include vinyl, allyl, isopropenyl, 2-butenyl, and the like.

As used herein, the term “alkynyl” refers to a monovalent substituent derived from a linear or branched unsaturated hydrocarbon having 2 to 40 carbon atoms and having one or more carbon-carbon triple bonds. Specific examples thereof may include ethynyl, 2-propynyl, and the like.

As used herein, the term “aryl” refers to a monovalent substituent derived from an aromatic hydrocarbon having 6 to 40 carbon atoms and having a single ring or a combination of two or more rings. Further, the aryl may also include a form in which two or more rings are simply pendant to or fused with each other. Specific examples of the aryl may include phenyl, naphthyl, phenanthryl, anthryl, and the like.

As used herein, the term “heteroaryl” refers to a monovalent substituent derived from a monoheterocyclic or polyheterocyclic aromatic hydrocarbon having 5 to 40 nuclear atoms. In this case, one or more carbons, specifically 1 to 3 carbons in the ring are substituted with a heteroatom such as N, O, S, or Se. Further, the heteroaryl may also include a form in which two or more rings are simply pendant to or fused with each other, and may also include a form in which two or more rings are fused with an aryl group. Examples of the heteroaryl may include a 6-membered monocyclic ring, such as pyridyl, pyrazinyl, pyrimidinyl, pyridazinyl, and triazinyl, a polycyclic ring, such as phenoxathienyl, indolizinyl, indolyl, purinyl, quinolyl, benzothiazole, and carbazolyl, and 2-furanyl, N-imidazolyl, 2-isoxazolyl, 2-pyridinyl, 2-pyrimidinyl, and the like.

As used herein, the term “alkoxy” refers to a monovalent substituent represented by R′O—, in which R′ is an alkyl having 1 to 40 carbon atoms, and may include a linear, branched, or cyclic structure. Examples of the alkyloxy may include methoxy, ethoxy, n-propoxy, 1-propoxy, t-butoxy, n-butoxy, pentoxy, and the like.

As used herein, the term “cycloalkyl” refers to a monovalent substituent derived from a monocyclic or polycyclic non-aromatic hydrocarbon having 3 to 40 carbon atoms. Examples of the cycloalkyl may include cyclopropyl, cyclopentyl, cyclohexyl, norbornyl, adamantine, and the like.

As used herein, the term “heterocycloalkyl” refers to a monovalent substituent derived from a non-aromatic hydrocarbon having 3 to 40 nuclear atoms, and one or more carbons, specifically 1 to 3 carbons in the ring are substituted with a hetero atom such as N, O, S, or Se. Examples of the heterocycloalkyl may include morpholine, piperazine, and the like.

As used herein, the term “fused ring” refers to a fused aliphatic ring, a fused aromatic ring, a fused heteroaliphatic ring, a fused heteroaromatic ring, or a combined form thereof.

Further, the present disclosure provides a salt, preferably a pharmaceutically acceptable salt of the compound represented by Chemical Formula 1.

Here, “pharmaceutically acceptable salt” means a salt suitable for use in contact with human and lower animal tissues without causing undue toxicity, irritation, allergic reaction, and the like within the scope of pure medical judgment. The pharmaceutically acceptable salts are well known in the art and are described in detail in, for example, the literature (S. M. Berge et al., J. Pharmaceutical Sciences, 66, 1, 1977). The salt may be prepared in situ during final isolation and purification of the compound of the present disclosure, or may be prepared by separately reacting with an inorganic base or an organic base. A specific example of the base addition salt form includes alkaline and alkaline earth metal salts such as ammonium salts, and salts of lithium, sodium, potassium, magnesium, calcium, and the like, salts with organic bases, for example, salts with primary, secondary and tertiary aliphatic and aromatic amines, for example, methylamine, ethylamine, propylamine, isopropylamine, four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline, benzathine, N-methyl-D-glucamine, 2-amino-2-(hydroxymethyl)-1,3-propanediol, hydrabamine sals, salts with amino acids such as arginine and lysine, and the like.

In addition, the present disclosure may include hydrates or solvates of the compound represented by Chemical Formula 1, and derivative compounds thereof. Solvents in the solvates are not particularly limited, and all typical solvents known in the art may be included.

The pharmaceutical composition according to the present disclosure may exhibit antiviral activity by inhibiting the penetration of viruses into host cells. That is, the viral infections may include infections by all pathogenic viruses known in the art to induce disease. Specifically, the viral infections may be caused by infection with viruses such as single-stranded DNA viruses, double-stranded DNA viruses, single-stranded RNA viruses, double-stranded RNA viruses and retroviruses. In an exemplary embodiment of the present disclosure, the viral infections may be caused by infection with single-stranded RNA viruses, more particularly single-stranded positive RNA viruses.

As an example, the single-stranded positive RNA viruses may be viruses belonging to the family Coronaviridae, Picornaviridae, Flaviviridae, Togaviridae, or Caliciviridae. Specifically, the single-stranded positive RNA virus may be coronavirus, norovirus, dengue virus, rhinovirus, West Nile virus, Zika virus, hepatitis C virus, polio virus, or rubella virus. The coronavirus may be SARS coronavirus (SARS-CoV), MERS coronavirus (MERS-CoV) or novel coronavirus (SARS-CoV-2). In an exemplary embodiment of the present disclosure, the coronavirus may be novel coronavirus.

The pharmaceutical composition according to the present disclosure may contain 10 to 95 wt % of the compound represented by Chemical Formula 1, or a pharmaceutically acceptable salt or solvate thereof, which is an active ingredient, based on the total weight of the composition. Further, the pharmaceutical composition of the present disclosure may further contain one or more active ingredients exhibiting the same or similar function in addition to the active ingredient.

The pharmaceutical composition of the present disclosure may contain a carrier, a diluent, an excipient, or a mixture thereof typically used in biological preparations. Any pharmaceutically acceptable carrier can be used as long as it is suitable for delivering the composition in vivo. Specifically, the carrier may be a compound described in Merck Index, 13th., Merck & Co. Inc., saline, sterilized water, Ringer's solution, a dextrose solution, a maltodextrin solution, glycerol, ethanol or a mixture thereof. Further, typical additives such as an antioxidant, a buffer, and a bacteriostatic agent may be added, if necessary.

When the composition is prepared, a commonly used diluent or excipient, such as a filler, an extender, a binder, a wetting agent, a disintegrant, and a surfactant may be added.

The pharmaceutical composition of the disclosure may be formulated into an oral or parenteral preparation. Oral preparations may include solid preparations and liquid preparations. The solid preparation may be a tablet, a pill, a powder, a granule, a capsule, or a troche, and the solid preparation may be prepared by adding at least one excipient to the composition. The excipient may be starch, calcium carbonate, sucrose, lactose, gelatin or a mixture thereof. In addition, the solid preparation may include a lubricant, examples of which include magnesium stearate, talc, and the like. Meanwhile, the liquid preparation may be a suspension, an internal solution, an emulsion or a syrup. In this case, the liquid preparation may contain an excipient such as a wetting agent, a sweetener, a fragrance, and a preservative.

The parenteral preparation may contain injections, suppositories, respiratory inhalation powders, aerosol formulations for spraying, powders, creams, and the like. The injection may include a sterilized aqueous solution, a non-aqueous solvent, a suspending agent, an emulsion, and the like. In this case, as the non-aqueous solvent or suspending agent, vegetable oils such as propylene glycol, polyethylene glycol and olive oil, and injectable esters such as ethyl oleate may be used.

Furthermore, the present disclosure provides a health functional food for preventing or ameliorating viral infections, containing the above-described compound represented by Chemical Formula 1, and a pharmaceutically acceptable salt or solvate thereof as an active ingredient.

The viral infection may have characteristics as described above.

The compound represented by Chemical Formula 1 of the present disclosure, and a pharmaceutically acceptable salt or solvate thereof may be directly added to food or used together with other food or food ingredients. In this case, the content of the added active ingredient may be determined according to the purpose, and generally may range from 0.01 to 90 parts by weight based on the weight of the entire food.

The form and type of health functional food are not particularly limited. Specifically, the health functional food may be in the form of tablet, capsule, powder, granule, liquid and pill. The functional health foods may contain various flavoring agents, sweeteners or natural carbohydrates as additional ingredients. The sweetener may be a natural or synthetic sweetener, and examples of the natural sweetener include thaumatin, stevia extract, and the like. Meanwhile, examples of the synthetic sweetener include saccharin, aspartame, and the like. In addition, the natural carbohydrates may be monosaccharides, disaccharides, polysaccharides, oligosaccharides, sugar alcohols, and the like.

The health functional food of the present disclosure may further include, in addition to the above-described additional ingredients, nutrients, vitamins, electrolytes, flavoring agents, colorants, pectic acid and salts thereof, alginic acid and salts thereof, organic acids, protective colloidal thickeners, pH adjusters, stabilizers, preservatives, glycerin, alcohols, and the like. These ingredients may be used either alone or in combinations thereof. The proportion of the additive may be selected in a range of 0.01 to 0.1 parts by weight per 100 parts by weight of the composition of the present disclosure.

In addition, the present disclosure provides a method for preventing, ameliorating or treating viral infections, the method including administering the above-described compound represented by Chemical Formula 1, and a pharmaceutically acceptable salt or solvate thereof to an individual.

The viral infection may have characteristics as described above.

The individual may be a mammal, specifically a human

The administering of the compound represented by Chemical Formula 1, and the pharmaceutically acceptable salt or solvate thereof to the individual may be administered orally or parenterally according to the desired method. Parenteral administration method may include intraperitoneal, rectal, subcutaneous, intravenous, intramuscular or intrathoracic injection modes.

Furthermore, the compound represented by Chemical Formula 1, and the pharmaceutically acceptable salt or solvate thereof may be administered in a pharmaceutically effective amount. This may vary depending on the type of disease, the severity, the activity of the drug, the patient's sensitivity to the drug, the time of administration, the route of administration, the duration of treatment, the drug being used simultaneously, and the like. However, for the desired effect, the amount of the active ingredient when administered may be 0.0001 to 1,000 mg/kg, specifically 0.001 to 500 mg/kg. The administration may be once or several times a day.

The compound represented by Chemical Formula 1, and the pharmaceutically acceptable salt or solvate thereof may be administered alone or in combination with other therapeutic agents. In combination administration, administration may be sequential or simultaneous.

Furthermore, the present disclosure provides a use of the above-described compound represented by Chemical Formula 1, and a pharmaceutically acceptable salt or solvate thereof for use in the preparation of a drug for preventing, ameliorating, or treating viral infections.

The viral infection may have characteristics as described above.

### MODE FOR INVENTION

Hereinafter, the present disclosure will be described in detail with reference to the following examples. However, the following examples are intended to illustrate the present disclosure, and the present disclosure is not limited thereto. Anything that has substantially the same configuration as the technical idea described in the claims of the present disclosure and achieves the same effect is included in the technical scope of the present disclosure.

### Preparation Example 1

### Synthesis of (E)-methyl-2-methoxy-5-(3-oxo-3-(2-oxo-5,6-dihydropyridin-1(2H)-yl)prop-1-en-1-yl)benzoate (Compound 1)

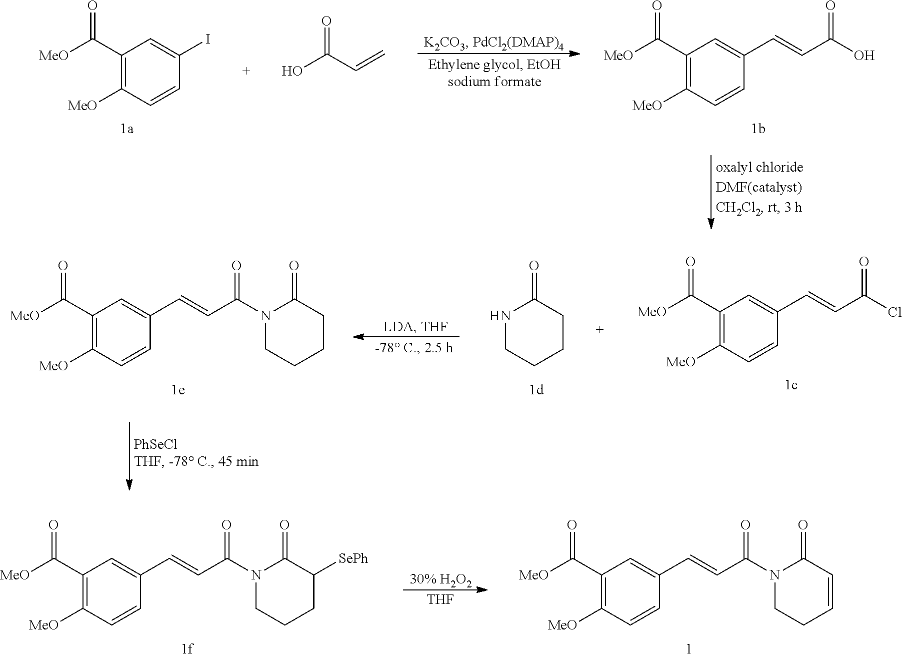

1-1. Preparation of (E)-methyl-2-methoxy-5-(3-oxo-3-(2-oxo-3-(phenylselanyl)piperidin-1-yl)prop-1-en-1-yl)benzoate (Compound 1f)

(E)-methyl-2-methoxy-5-(3-oxo-3-(2-oxopiperidin-1-yl)prop-1-en-1-yl)benzoate (Compound 1e) (0.22 g, 0.68 mmol) was put into a 25-ml round bottom flask under an argon atmosphere, tetrahydrofuran (3.70 ml) was charged, then the temperature was lowered to −78° C., LDA (0.45 ml, 0.88 mmol, 2.0 M solution) was added dropwise thereto, and the resulting mixture was stirred for 45 minutes. Phenylselenyl chloride (0.14 g, 0.75 mmol) was dissolved in tetrahydrofuran (3.75 ml) and slowly added at −78° C., and the resulting solution was stirred for 4.5 hours. When the reaction was completed, water was added to decompose the remaining LDA, and the mixture was further stirred at 0° C. for 15 minutes and extracted twice with methane dichloride. Hereinafter, the organic layer was washed once with a saturated sodium chloride solution, dried over anhydrous sodium sulfate, evaporated under reduced pressure, and then separated by silica gel chromatography (ethyl acetate/hexane=1/4 to 1/3) to obtain (E)-methyl-2-methoxy-5-(3-oxo-3-(2-oxo-3-(phenylselanyl)piperidin-1-yl)prop-1-en-yl)benzoate (Compound 10 (100 mg, 31.2%) as a white solid.

¹H NMR(400 MHz, Chloroform-d) δ 7.95 (d, J=2.3 Hz, 1H), 7.72-7.68 (m, 2H), 7.67-7.60 (m, 2H), 7.38-7.31 (m, 3H), 7.05 (d, J=15.6 Hz, 1H), 6.98 (d, J=8.8 Hz, 1H), 4.11 (t, J=5.3 Hz, 1H), 3.95 (s, 3H), 3.92 (s, 3H), 3.71 (ddd, J=13.7, 9.2, 4.6 Hz, 1H), 2.32 (tq, J=10.5, 5.4 Hz, 1H), 2.19-2.05 (m, 2H), 1.26 (d, J=2.7 Hz, 2H).

1-2. (E)-methyl-2-methoxy-5-(3-oxo-3-(2-oxo-5,6-dihydropyridin-1(2H)-yl)prop-1-en-1-yl)benzoate (Compound 1)

(E)-methyl-2-methoxy-5-(3-oxo-3-(2-oxo-3-(phenylselanyl)piperidin-1-yl)prop-1-en-1-yl)benzoate (Compound 1f) (0.10 g, 0.21 mmol) prepared in Preparation Example 1-1 was put into a 25-ml round bottom flask under an argon atmosphere, tetrahydrofuran (2.00 ml) was charged, then the temperature was lowered to 0° C., hydrogen peroxide (0.05 ml, 0.59 mmol, 30% solution) was added dropwise thereto, and the resulting mixture was stirred for 15 minutes. Thereafter, the temperature was raised to room temperature, the mixture was stirred for another 30 minutes, and when the reaction was completed, a saturated sodium bicarbonate solution was added thereto, the resulting mixture was extracted twice with methane dichloride, and the organic layer was washed once with a saturated sodium chloride solution. The organic layer was dried over anhydrous sodium sulfate, evaporated under reduced pressure, and then separated by silica gel chromatography (ethyl acetate/hexane=1/1.5) to obtain (E)-methyl-2-methoxy-5-(3-oxo-3-(2-oxo-5,6-dihydropyridin-1(2H)-yl)prop-1-en-1-yl)benzoate (Compound 1) (40 mg, 60.6%) as a white solid.

¹H NMR (400 MHz, Chloroform-d) δ 8.01 (d, J=2.4 Hz, 1H), 7.74-7.66 (m, 2H), 7.43 (d, J=15.7 Hz, 1H), 7.00-6.92 (m, 2H), 6.05 (dt, J=9.6, 1.8 Hz, 1H), 4.04 (t, J=6.5 Hz, 2H), 3.94 (s, 3H), 3.90 (s, 3H), 2.48 (tdd, J=6.3, 4.2, 1.9 Hz, 2H).

¹³C NMR (101 MHz, Chloroform-d) δ 168.92, 166.08, 165.86, 160.38, 145.51, 142.33, 133.42, 131.75, 127.39, 125.87, 120.69, 120.48, 112.24, 56.22, 52.20, 41.65, 24.81.

### Preparation Example 2

### Synthesis of (E)-methyl-2-methoxy-5-(3-(4-methylpiperazin-1-yl)-3-oxoprop-1-en-1-yl)benzoate (Compound 2)

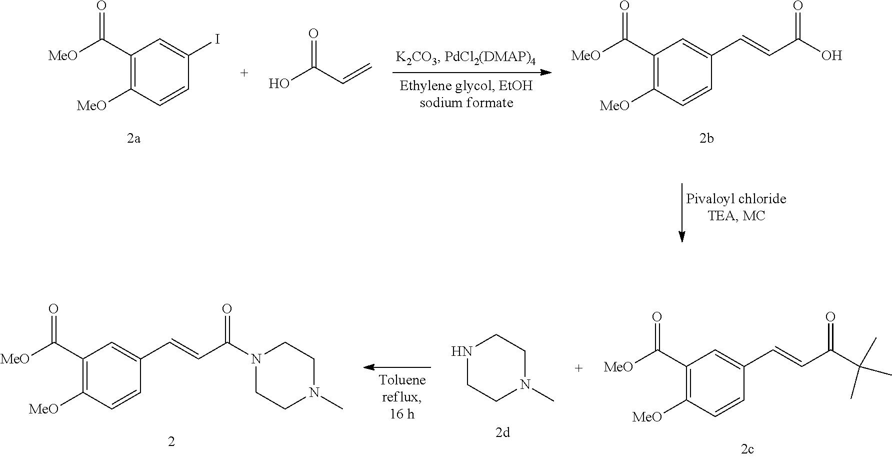

(E)-3-(4-methoxy-3-(methoxycarbonyl)phenyl) acrylic acid (Compound 2b) (0.20 g, 0.85 mmol) was put into a 25-ml round bottom flask under an argon atmosphere, dichloromethane (12.0 ml) was charged, then the temperature was lowered to 0° C., triethylamine (0.18 ml, 1.28 mmol) and pivaloyl chloride (0.12 ml, 0.94 mmol) were slowly added thereto, and the resulting mixture was stirred at the same temperature for 45 minutes. Methyl piperazine (2d) (0.15 ml, 1.28 mmol) was then slowly added to the reaction mixture, and the resulting mixture was stirred at room temperature for 2 hours. When the reaction was completed, a saturated sodium bicarbonate solution was added to the mixture to terminate the reaction, and water was added to dilute the mixture. The aqueous layer was extracted three times with methane dichloride, the organic layer was washed twice with a saturated sodium chloride solution and dried over anhydrous sodium sulfate, the solvent of the mixture was removed by distillation under reduced pressure, and then the residue was separated by silica gel chromatography (methanol/methane dichloride=1/25) to obtain (E)-methyl-2-methoxy-5-(3-(4-methylpiperazin-1-yl)-3-oxoprop-1-en-1-yl)benzo ate (Compound 2) (250 mg, 61.4%) as a pale yellow oil.

¹H NMR (400 MHz, Chloroform-d) δ 8.00 (d, J=2.3 Hz, 1H), 7.66-7.56 (m, 2H), 6.98 (d, J=8.7 Hz, 1H), 6.80 (d, J=15.4 Hz, 1H), 3.94 (s, 3H), 3.92 (s, 3H), 3.73-3.62 (m, 4H), 2.45 (t, J=5.1 Hz, 4H), 2.33 (s, 3H).

### Preparation Example 3

### Synthesis of (E)-methyl-5-(3-(3-chloro-2-oxo-5,6-dihydropyridin-1(2H)-yl)-3-oxoprop-1-en-1-yl)-2-methoxybenzoate (Compound 3)

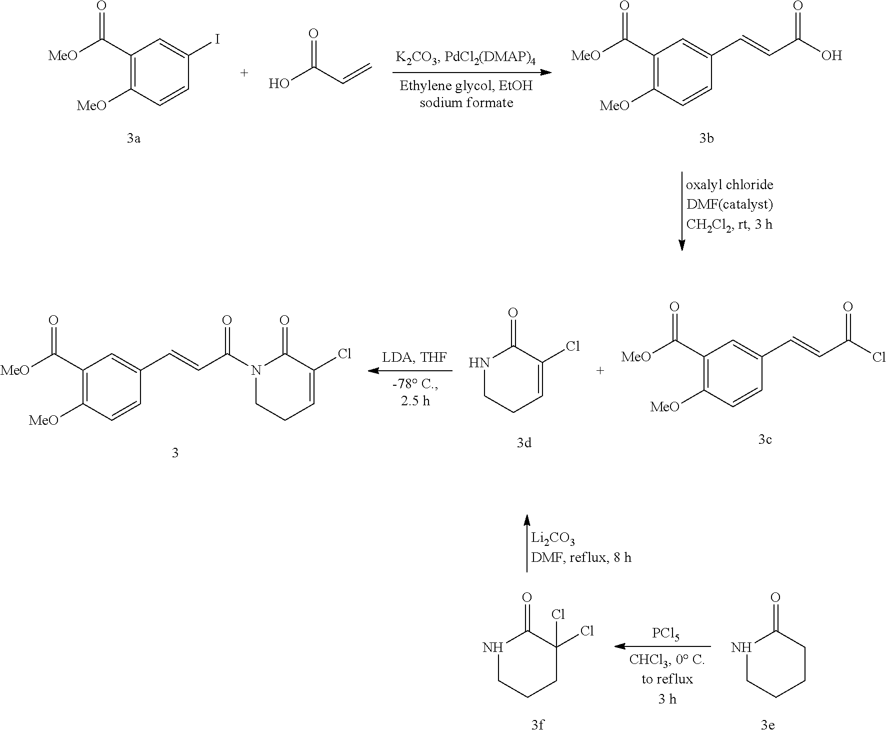

3-1. Preparation of 3,3-dichloropiperidin-2-one (Compound 3f)

Piperidine-2-one (Compound 3e) (2.00 g, 20.18 mmol) was put into a 100-ml round bottom flask under an argon atmosphere, chloroform (20.00 ml) was charged, then the temperature was lowered to 0° C., phosphorus pentachloride (12.80 g, 61.33 mmol) was slowly added thereto for 10 minutes, and the resulting mixture was stirred under reflux at 60° C. When the reaction was completed, the temperature was lowered to room temperature, and the mixture was put into ice water. The aqueous layer was extracted three times with methane dichloride, the organic layer was washed once with a saturated sodium chloride solution, dried over anhydrous magnesium sulfate and evaporated under reduced pressure, and then 3,3-dichloropiperidine-2-one (Compound 3f) (4.00 g) as a white solid without further purification.

¹H NMR (400 MHz, Chloroform-d) δ 6.18 (s, 1H), 3.44 (td, J=6.2, 2.4 Hz, 2H), 2.82-2.74 (m, 2H), 2.15-2.05 (m, 2H).

3-2. Preparation of 3-chloro-5,6-dihydropyridin-2(1H)-one (Compound 3d)

3,3-dichloropiperidine-2-one (Compound 3f) (4.00 g, 24.04 mmol) prepared in Preparation Example 3-1 was put into a 50-ml round bottom flask under an argon atmosphere, dimethylformamide (12.00 ml) was charged, then lithium carbonate (3.70 g, 49.30 mmol) was added thereto, and the resulting mixture was stirred under heating at 120° C. for 7 hours. When the reaction was completed, the temperature was lowered to room temperature, and the mixture was put into ice water. The aqueous layer was extracted three times with methane dichloride, and the organic layer was washed once with a saturated sodium chloride solution, dried over anhydrous magnesium sulfate, and then separated by silica gel chromatography (ethyl acetate/hexane=1/1.5 to 1/1) to obtain 3-chloro-5,6-dihydropyridin-2(1H)-one (Compound 3d) (0.55 g, 17.6%) as a brown solid.

¹H NMR (400 MHz, Chloroform-d) δ 6.78 (t, J=4.6 Hz, 1H), 6.70 (s, 1H), 3.53-3.43 (m, 2H), 2.48 (td, J=7.1, 4.5 Hz, 2H).

3-3. Preparation of (E)-methyl-5-(3-(3-chloro-2-oxo-5,6-dihydropyridin-1(2H)-yl)-3-oxoprop-1-en-1-yl)-2-methoxybenzoate (Compound 3)

3-chloro-5,6-dihydropyridin-2(1h)-one (Compound 3d) (0.10 g, 0.77 mmol) prepared in Preparation Example 3-2 was put into a 25-ml round bottom flask under an argon atmosphere, tetrahydrofuran (1.50 ml) was charged, then the temperature was lowered to −78° C., LDA (0.40 ml, 0.77 mmol, 2.0 M solution) was added thereto, and the resulting mixture was stirred for 45 minutes. (E)-methyl 5-(3-chloro-3-oxopro-1-pen-1-yl)-2-methoxybenzoate (Compound 3c) (0.16 g, 0.64 mmol) was dissolved in tetrahydrofuran (1.50 ml), the resulting solution was slowly added thereto at −78° C., and the resulting mixture was stirred for 1 hour. When the reaction was completed, the remaining LDA was decomposed with 1 N hydrochloric acid, and then extracted twice with ethyl acetate, and the ethyl acetate layer was washed once with a saturated sodium chloride solution. Thereafter, the ethyl acetate layer was dried over anhydrous sodium sulfate, distilled under reduced pressure, and then separated by column chromatography (ethyl acetate/hexane=1/2 to 1/1) to obtain (E)-methyl-5-(3-(3-chloro-2-oxo-5,6-dihydroxypyridin-1(2H)-yl)-3-oxoprop-1-en-1-yl)-2-methoxybenzo ate (Compound 3) (98 mg, 43.8%) as a white solid.

¹H NMR (400 MHz, Chloroform-d) δ 8.02 (d, J=2.3 Hz, 1H), 7.74 (d, J=15.3 Hz, 1H), 7.71-7.69 (m, 1H), 7.43 (d, J=15.6 Hz, 1H), 7.09 (t, J=4.6 Hz, 1H), 6.98 (d, J=8.7 Hz, 1H), 4.09 (t, J=6.5 Hz, 2H), 3.95 (s, 3H), 3.91 (s, 3H), 2.57 (td, J=6.5, 4.6 Hz, 2H).

¹³C NMR (101 MHz, Chloroform-d) δ 168.52, 166.01, 161.46, 160.58, 143.43, 141.09, 133.53, 131.84, 128.29, 127.13, 120.59, 119.91, 112.27, 56.24, 52.23, 41.76, 25.32.

### Preparation Example 4

### Synthesis of (E)-methyl-2-methoxy-5-(3-oxo-3-(2-oxo-2,5-dihydro-1H-pyrrol-1-yl)prop-1-en-1-yl)benzoate (Compound 5)

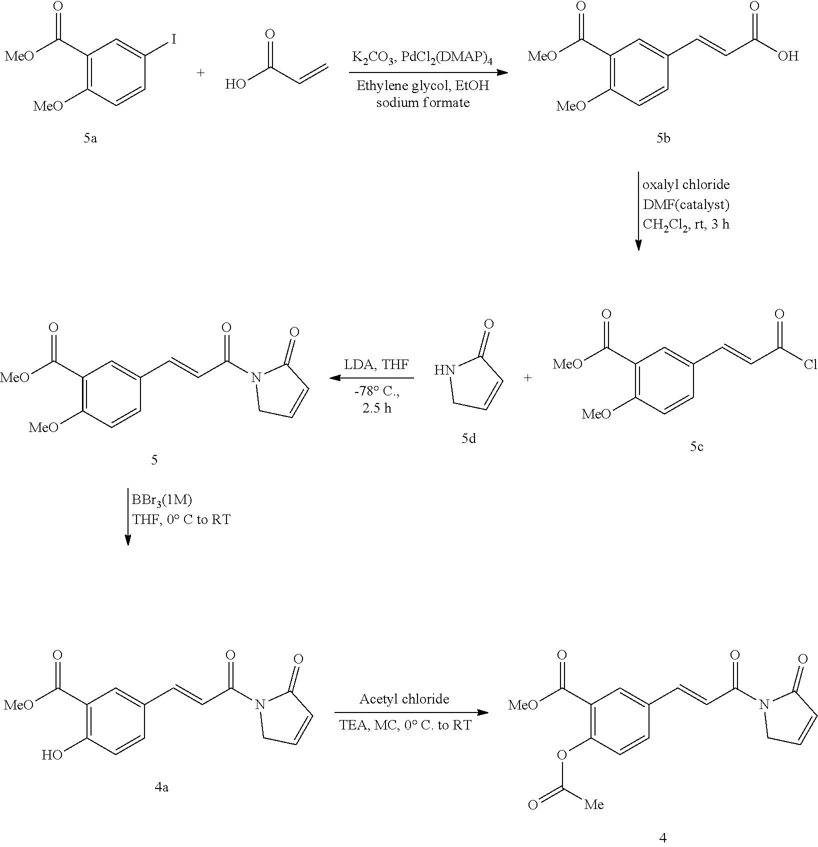

4-1. Preparation of (E)-3-(4-methoxy-3-(methoxycarbonyl)phenyl)acrylic acid (Compound 5b)

A mixture obtained by stirring an aqueous potassium carbonate solution (1.06 g/2.50 ml of distilled water, 7.64 mmol), ethylene glycol (2.50 ml), acrylic acid (0.48 ml, 6.76 mmol), ethanol (0.25 ml), sodium formate (14.0 mg, 0.20 mmol) and PdCl₂(DMAP)₄ (13.0 mg, 0.02 mmol) was added to methyl-5-iodo-2-methoxybenzoate (Compound 5a) (1.50 g, 5.14 mmol). The mixture was carefully stirred for 5 minutes and then refluxed until a black palladium precipitate was formed. The reaction mixture was cooled to room temperature, and then titrated to pH 1 using a 1 N hydrochloric acid solution. The resulting solid was filtered, then washed three times with water, and dried to obtain a compound (E)-3-(4-methoxy-3-(methoxycarbonyl)phenyl)acrylic acid (Compound 5b) (1.02 g, 83.4%) as a white solid.

¹H NMR (400 MHz, DMSO-d6) δ 12.30 (s, 1H), 7.94-7.87 (m, 2H), 7.56 (d, J=16.0 Hz, 1H), 7.20 (d, J=8.7 Hz, 1H), 6.45 (d, J=15.9 Hz, 1H), 3.86 (s, 3H), 3.80 (s, 3H), 2.09 (s, 1H).

¹³ C NMR (101 MHz, DMSO-d6) δ 167.60, 165.81, 159.23, 142.53, 133.05, 130.48, 126.40, 120.66, 117.98, 112.96, 60.19, 56.08, 52.02.

### 4-2. Preparation of 1H-pyrrol-2(5H)-one (Compound 5d)

After pyrrole (5.0 ml, 72.0 mmol) and barium carbonate (1.50 g, 7.60 mmol) were put into a round bottom flask under an argon atmosphere, distilled water (0.30 1) was charged, and 30% hydrogen peroxide (9.0 mL) was added dropwise thereto. The flask was equipped with a condenser, the mixture was refluxed for 5 hours, and then the temperature was lowered to room temperature, and the remaining hydrogen peroxide was removed using a 10% sodium nitrite solution. After stirring until foaming ceased, the mixture was filtered through filter paper, and the solvent was distilled under reduced pressure. After a reddish-black solid was dissolved in 1,4-dioxane (50.0 ml), the resulting solution was again filtered through filter paper, the filtered product was washed with 1,4-dioxane (50.0 ml), then dried over anhydrous sodium sulfate, and distilled under reduced pressure to obtain 1H-pyrrol-2(5H)-one (Compound 5d) (1.42 g, 23.9%) as a reddish-black liquid.

¹H NMR (400 MHz, CDCl₃): δ 9.27 (1H, br s), 7.16-7.19 (1H, dt, J=6.0, 1.8 Hz), 6.17-6.19(1H, d, J=6.0 Hz). 4.50 (2H, d, J=1.8 Hz).

¹³C NMR (101 MHz, CDCl₃): δ 175.5, 146.2, 127.8, 49.2.

4-3. Preparation of (E)-methyl-2-methoxy-5-(3-oxo-3-(2-oxo-2,5-dihydro-1H-pyrrol-1-yl)prop-1-en-1-yl)benzoate (Compound 5)

1H-pyrrol-2(5H)-one (Compound 5d) (0.21 g, 2.55 mmol) prepared in Preparation Example 4-2 was put into a 50-ml round bottom flask under an argon atmosphere, tetrahydrofuran (3.30 ml) was charged, then the temperature was lowered to −78° C., LDA (1.30 ml, 2.55 mmol, 2.0 M solution) was added thereto, and the resulting mixture was stirred for 45 minutes. (E)-methyl-5-(3-chloro-3-oxopro-1-pen-1-yl)-2-methoxybenzoate (Compound 5c) (0.54 g, 2.12 mmol) was dissolved in tetrahydrofuran (2.70 ml), the resulting solution was slowly added thereto at −78° C., and the resulting mixture was stirred for 1 hour. When the reaction was completed, the remaining LDA was decomposed with 1 N hydrochloric acid, and then extracted with ethyl acetate, and the ethyl acetate layer was washed with a saturated sodium chloride solution. The ethyl acetate layer was dried over anhydrous sodium sulfate, distilled under reduced pressure, and then separated by column chromatography (ethyl acetate/hexane=1/2 to 1/1) to obtain (E)-methyl-2-methoxy-5-(3-oxo-3-(2-oxo-2,5-dihydro-1H-pyrrol-1-yl)prop-1-en-1-yl)benzoate (Compound 5) (75 mg, 30.0%) as a yellow solid.

¹H NMR (400 MHz, CDCl₃) δ 8.06 (d, J=2.3 Hz, 1H), 7.96 (d, J=15.7 Hz, 1H), 7.86 (d, J=15.7 Hz, 1H), 7.78 (dd, J=8.7, 2.3 Hz, 1H), 7.35 (dt, J=6.0, 2.1 Hz, 1H), 7.01 (d, J=8.8 Hz, 1H), 6.22 (dt, J=6.0, 1.9 Hz, 1H), 4.54 (t, J=2.0 Hz, 2H), 3.96 (s, 3H), 3.92 (s, 3H).

¹³C NMR (101 MHz, Chloroform-d) δ 170.21, 166.02, 165.20, 160.68, 146.84, 144.71, 133.49, 132.25, 127.87, 127.16, 120.56, 117.39, 112.32, 56.25, 52.25, 51.11.

### Preparation Example 5

### Synthesis of (E)-methyl-2-acetoxy-5-(3-oxo-3-(2-oxo-2,5-dihydro-1H-pyrrol-1-yl)prop-1-en-1-yl)benzoate (Compound 4)

5-1. Preparation of (E)-methyl-2-hydroxy-5-(3-oxo-3-(2-oxo-2,5-dihydro-1H-pyrrol-1-yl)prop-1-en-1-yl)benzoate (Compound 4a)

(E)-methyl-2-methoxy-5-(3-oxo-3-(2-oxo-2,5-dihydro-1-pyrrol-1-en-1-yl)prop-1-en-1-yl)benzoate (Compound 5) (70 mg, 0.23 mmol) prepared in Preparation Example 4 and tetrahydrofuran (4.0 ml) were put into a round bottom flask under an argon atmosphere, the temperature was lowered to 0° C., and after 10 minutes, a tribromoboron solution (a 1.0 M methane dichloride solution, 0.70 ml, 0.69 mmol) was slowly added thereto. The reaction temperature was gradually raised to room temperature while stirring the resulting mixture for 4 hours. It was confirmed by TLC whether the reaction was completed, and when the reaction was completed, the reaction temperature was again lowered to 0° C., and the reaction was terminated using 0.50 ml of a 1 N hydrochloric acid solution. After the mixture was stirred at the same temperature for another 10 minutes, the resulting product was extracted three times with methane dichloride, and the organic layer was washed once with a saturated sodium chloride solution. Thereafter, the organic layer was dried over anhydrous sodium sulfate, evaporated under reduced pressure, and then separated by silica gel chromatography (ethyl acetate/hexane=1/2 to 1/1) to obtain (E)-methyl-2-hydroxy-5-(3-oxo-3-(2-oxo-2,5-dihydro-1H-pyrrol-1-yl)prop-1-en-1-yl)benzoate (Compound 4a) (10 mg, 15.2%) as a brown solid.

¹H NMR (400 MHz, Chloroform-d) δ 11.05 (s, 1H), 8.11 (d, J=2.3 Hz, 1H), 7.95 (d, J=15.8 Hz, 1H), 7.85 (d, J=15.8 Hz, 1H), 7.80 (dd, J=8.7, 2.3 Hz, 1H), 7.35 (dt, J=6.0, 2.1 Hz, 1H), 7.03 (d, J=8.7 Hz, 1H), 6.22 (dt, J=6.0, 1.9 Hz, 1H), 4.54 (t, J=1.9 Hz, 2H), 3.99 (s, 3H).

5-2. Preparation of (E)-methyl-2-acetoxy-5-(3-oxo-3-(2-oxo-2,5-dihydro-1H-pyrrol-1-yl)prop-1-en-1-yl)benzoate (Compound 4)

(E)-methyl-2-hydroxy-5-(3-oxo-3-(2-oxo-2,5-dihydroxy-1H-pyrrol-1-yl)prop-1-en-1-yl)benzoate prepared in Preparation Example 5-1 (Compound 4a) (10 mg, 0.04 mmol) and methane dichloride (1.0 ml) were put into a round bottom flask under an argon atmosphere, and the temperature was lowered to 0° C. After 10 minutes, triethylamine (11.2 μl, 0.08 mmol) was added thereto, and the resulting mixture was stirred for 5 minutes. Acetate chloride (5.70 μl, 0.08 mmol) was slowly added thereto at the same temperature, and the resulting mixture was stirred for 1 hour while maintaining the temperature at 0° C. It was confirmed by TLC whether the reaction was completed, and after the reaction was completed, the reaction was terminated using 1.0 ml of distilled water, the resulting product was extracted three times with methane dichloride, and the organic layer was washed once with a saturated sodium chloride solution. The organic layer was dried over anhydrous sodium sulfate, evaporated under reduced pressure, and then separated by silica gel chromatography (ethyl acetate/hexane=1/2 to 1/1) to obtain (E)-methyl-2-acetoxy-5-(3-oxo-3-(2-oxo-2,5-dihydro-1H-pyrrol-1-yl)prop-1-en-1-yl)benzo ate (Compound 4) (5.0 mg, 43.5%) as a yellow solid.

¹H NMR (400 MHz, Chloroform-d) δ 8.24 (d, J=2.2 Hz, 1H), 8.05 (d, J=15.8 Hz, 1H), 7.89 (d, J=15.8 Hz, 1H), 7.85 (dd, J=8.4, 2.3 Hz, 1H), 7.37 (dt, J=6.2, 2.1 Hz, 1H), 7.16 (d, J=8.4 Hz, 1H), 6.23 (dt, J=6.1, 1.9 Hz, 1H), 4.55 (t, J=2.0 Hz, 2H), 3.91 (s, 3H), 2.37 (s, 3H).

### Preparation Example 6

### Synthesis of (E)-methyl-5-(3-acetamido-3-oxoprop-1-en-1-yl)-2-methoxybenzoate (Compound 6)

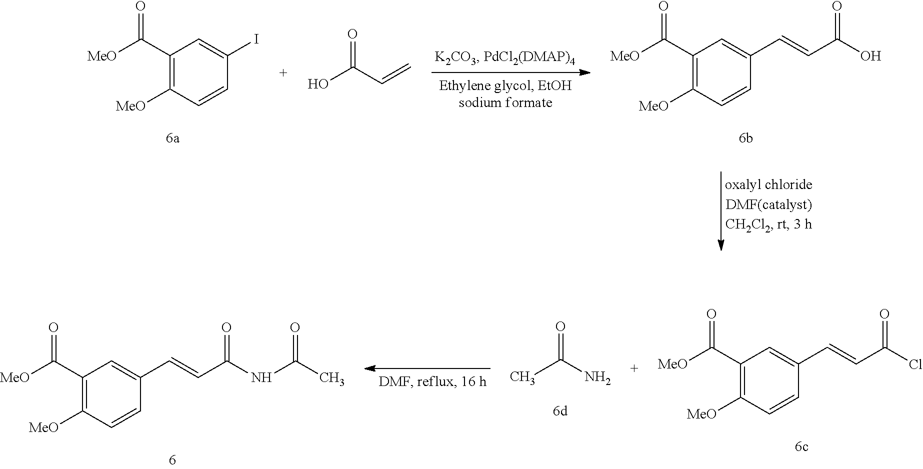

(E)-methyl 5-(3-chloro-3-oxopro-1-pen-1-yl) -2-methoxybenzoate (Compound 6c) (0.22 g, 0.85 mmol) was put into a 25-ml round bottom flask under an argon atmosphere, dimethylformamide (12.00 ml) was charged, then the acetamide (0.06 ml, 1.23 mmol) was added thereto, and the resulting mixture was stirred under reflux for 16 hours. When the reaction was completed, the temperature was lowered to room temperature, then the solvent of the mixture was removed by distillation under reduced pressure, and then the residue was separated by silica gel chromatography (ethyl acetate/hexane=1/2 to 1/1) to obtain (E)-methyl-5-(3-acetamido-3-oxoprop-1-en-1-yl)-2-methoxybenzoate (Compound 6) (30 mg, 12.7%) as a white solid.

¹H NMR (400 MHz, Chloroform-d) δ 8.11 (s, 1H), 8.03 (d, J=2.4 Hz, 1H), 7.77 (d, J=15.6 Hz, 1H), 7.68 (dd, J=8.8, 2.5 Hz, 1H), 7.01 (d, J=8.7 Hz, 1H), 6.79 (d, J=15.7 Hz, 1H), 3.96 (s, 3H), 3.92 (s, 3H), 2.45 (s, 3H).

### Preparation Example 7

### Synthesis of (E)-methyl-5-(3-(allylamino)-3-oxoprop-1-en-1-yl)-2-methoxybenzoate (Compound 7)

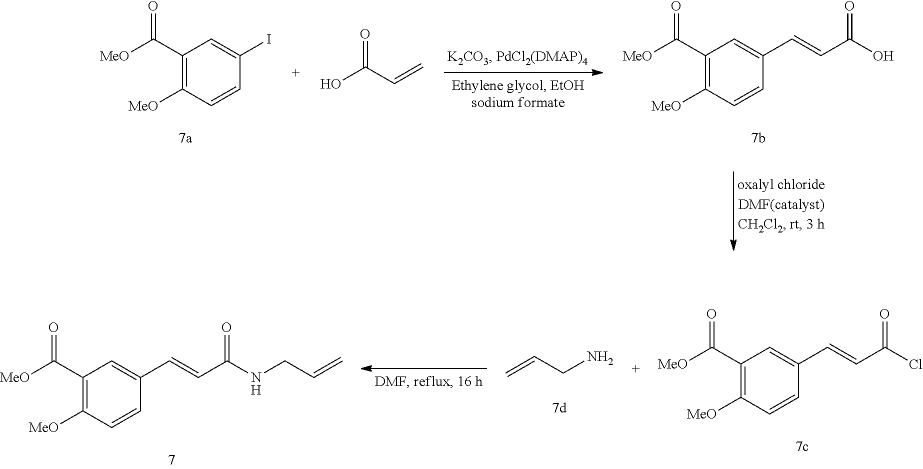

(E)-methyl 5-(3-chloro-3-oxopro-1-pen-1-yl) -2-methoxybenzoate (Compound 7c) (0.22 g, 0.85 mmol) was put into a 25-ml round bottom flask under an argon atmosphere, diethyl ether (2.50 ml) was charged, then the temperature was lowered to 0° C., and the resulting mixture was stirred at the same temperature for 10 minutes while slowly adding allylamine (Compound 7d) (0.64 ml, 8.50 mmol) thereto. When the reaction was completed, the solvent of the mixture was removed by distillation under reduced pressure, and then the residue was separated by silica gel chromatography (ethyl acetate/hexane=1/3 to 1/2) to obtain (E)-methyl-5-(3-(allylamino)-3-oxoprop-1-en-1-yl)-2-methoxybenzoate (Compound 7) (172 mg, 73.5%) as a white solid.

¹H NMR (400 MHz, Chloroform-d) δ 7.99 (d, J=2.4 Hz, 1H), 7.63-7.56 (m, 2H), 6.98 (d, J=8.7 Hz, 1H), 6.34 (d, J=15.6 Hz, 1H), 5.90 (ddt, J=17.1, 10.2, 5.6 Hz, 1H), 5.63 (s, 1H), 5.29-5.13 (m, 2H), 4.03 (tt, J=5.8, 1.5 Hz, 2H), 3.94 (s, 3H), 3.91 (s, 3H).

### Preparation Example 8

### Synthesis of (E)-methyl-2-methoxy-5-(3-oxo-3-(2-oxopiperidin-1-yl)prop-1-en-1-yl)benzoate (Compound 8)

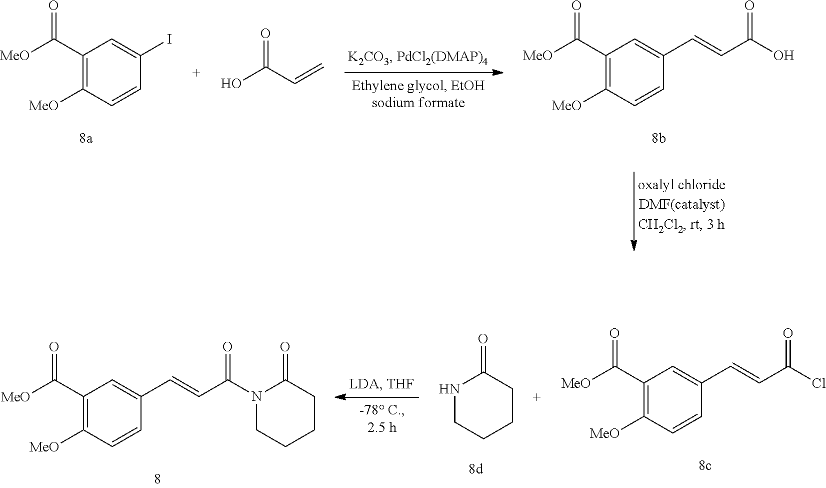

2-piperidinone (Compound 8d) (0.15 g, 1.53 mmol) was put into a 25-ml round bottom flask under an argon atmosphere, tetrahydrofuran (2.00 ml) was charged, then the temperature was lowered to −78° C., LDA (0.80 ml, 1.53 mmol, 2.0 M solution) was added thereto, and the resulting mixture was stirred for 45 minutes. (E)-methyl-5-(3-chloro-3-oxopro-1-pen-1-yl)-2-methoxybenzoate (Compound 8c) (0.32 g, 1.27 mmol) was dissolved in tetrahydrofuran (1.60 ml), the resulting solution was slowly added thereto at −78° C., and the resulting mixture was stirred for 1 hour. When the reaction was completed, the remaining LDA was decomposed with 1 N hydrochloric acid, and then extracted twice with ethyl acetate, and the ethyl acetate layer was washed once with a saturated sodium chloride solution. The ethyl acetate layer was dried over anhydrous sodium sulfate, distilled under reduced pressure, and then separated by column chromatography (ethyl acetate/hexane=1/2.5 to 1/2) to obtain (E)-methyl-2-methoxy-5-(3-oxo-3-(2-oxopiperidin-1-yl)prop-1-en-1-yl)benzo ate (Compound 8) (215 mg, 53.5%) as a white solid.

¹H NMR (400 MHz, Chloroform-d) δ 8.00 (d, J=2.3 Hz, 1H), 7.69-7.64 (m, 2H), 7.36 (d, J=15.6 Hz, 1H), 6.98 (d, J=8.7 Hz, 1H), 3.94 (s, 3H), 3.90 (s, 3H), 3.80 (td, J=5.2, 4.2, 2.2 Hz, 2H), 2.65-2.57 (m, 2H), 1.95-1.84 (m, 4H).

### Preparation Example 9

### Synthesis of (E)-methyl-5-(3-(3-bromo-2-oxo-5,6-dihydropyridin-1(2H)-yl)-3-oxoprop-1-en-1-yl)-2-methoxybenzoate (Compound 9)

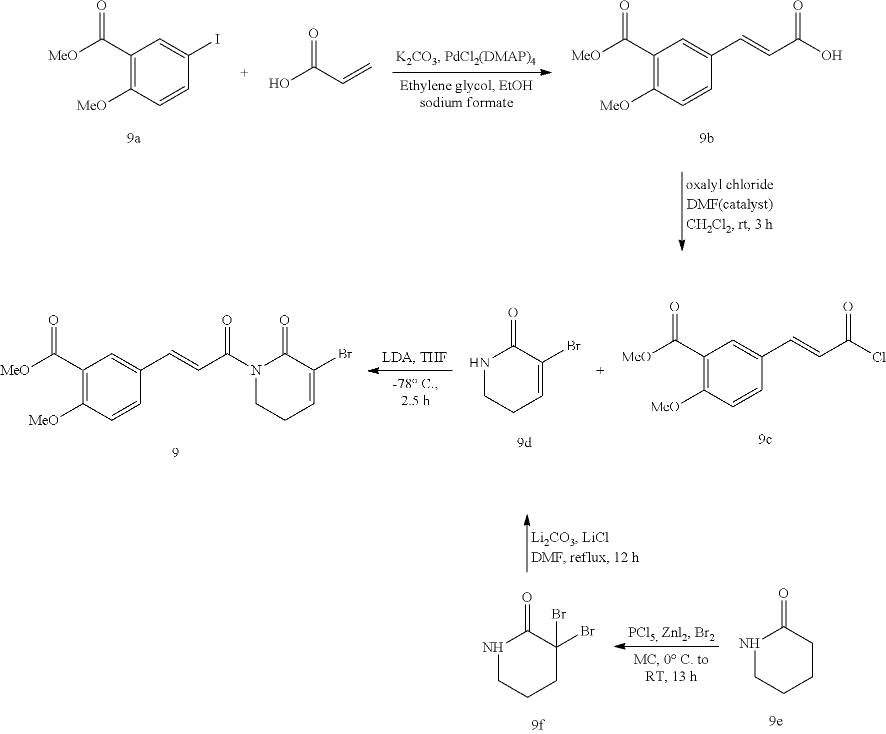

9-1. Preparation of 3,3-dibromopiperidin-2-one (Compound 9f) Piperidine-2-one (Compound 9e) (2.00 g, 20.18 mmol) was put into a 100-ml round bottom flask under an argon atmosphere, dichloromethane (40.00 ml) was charged, then the temperature was lowered to 0° C., phosphorus pentachloride (8.40 g, 40.36 mmol) was slowly added thereto for 5 minutes, and the resulting mixture was stirred for 5 minutes. After zinc iodide (0.20 g, 0.61 mmol) was added thereto at the same temperature, the temperature was raised to room temperature, and the resulting mixture was stirred for 1 hour. After 1 hour, heteroatom bromine (2.20 ml, 40.36 mmol) diluted in dichloromethine (20.00 ml) was slowly added thereto, and then, the resulting mixture was stirred at the same temperature for 12 hours. When the reaction was completed, the temperature was lowered to room temperature, and the mixture was put into ice water. The aqueous layer was extracted five times with methane dichloride and the organic layer was washed once with a saturated sodium chloride solution. The organic layer was dried over anhydrous magnesium sulfate, the solvent of the mixture was removed by distillation under reduced pressure, and then the residue was separated by silica gel chromatography (ethyl acetate/hexane=1/2.5 to 1/1) to obtain 3,3-dibromopiperidin-2-one (Compound 90(1.32 g, 25.5%) as a white solid.

¹H NMR (400 MHz, Chloroform-d) δ 6.17 (s, 1H), 3.47 (td, J=6.2, 2.4 Hz, 2H), 3.02-2.95 (m, 2H), 2.08-2.01 (m, 2H).

9-2. Preparation of 3-bromo-5,6-dihydropyridin-2(1H)-one (Compound 9d)

3,3-dibromopiperidin-2-one (Compound 9f) (1.32 g, 5.14 mmol) prepared in Preparation Example 9-1 was put into a 50-ml round bottom flask under an argon atmosphere, dimethylformamide (10.00 ml) was charged, then lithium carbonate (0.72 g, 9.80 mmol) and lithium chloride (0.22 g, 5.24 mmol) were added thereto, and the resulting mixture was stirred under heating at 120° C. for 13 hours. When the reaction was completed, the temperature was lowered to room temperature, and the mixture was put into ice water. The aqueous layer was extracted three times with methane dichloride and the organic layer was washed once with a saturated sodium chloride solution. The organic layer dried over anhydrous magnesium sulfate, and then separated by silica gel chromatography (ethyl acetate/hexane=1/1.5 to 1/1) to obtain 3-bromo-5,6-dihydropyridin-2(1H)-one (Compound 9d) (0.36 g, 39.9%) as a light brown solid.

¹H NMR (400 MHz, Chloroform-d) δ 7.05 (t, J=4.6 Hz, 1H), 6.96 (s, 1H), 3.49 (td, J=7.1, 2.8 Hz, 2H), 2.43 (td, J=7.1, 4.5 Hz, 2H)

9-3. Preparation of (E)-methyl-5-(3-(3-bromo-2-oxo-5,6-dihydropyridin-1(2H)-yl)-3-oxoprop-1-en-1-yl)-2-methoxybenzo ate (Compound 9)

3-bromo-5,6-dihydropyridin-2(1H)-one (Compound 9d) (0.14 g, 0.77 mmol) prepared in Preparation Example 9-2 was put into a 25-ml round bottom flask under an argon atmosphere, tetrahydrofuran (1.50 ml) was charged, then the temperature was lowered to −78° C., LDA (0.40 ml, 0.77 mmol, 2.0 M solution) was added thereto, and the resulting mixture was stirred for 45 minutes. (E)-methyl 5-(3-chloro-3-oxopro-1-pen-1-yl)-2-methoxybenzoate (Compound 9c) (0.16 g, 0.64 mmol) was dissolved in tetrahydrofuran (1.50 ml), the resulting solution was slowly added thereto at −78° C., and the resulting mixture was stirred for 1 hour. When the reaction was completed, the remaining LDA was decomposed with 1 N hydrochloric acid, and then extracted twice with ethyl acetate, and the ethyl acetate layer was washed once with a saturated sodium chloride solution. The ethyl acetate layer was dried over anhydrous sodium sulfate, distilled under reduced pressure, and then separated by column chromatography (ethyl acetate/hexane=1/2 to 1/1) to obtain (E)-methyl-5-(3-(3-bromo-2-oxo-5,6-dihydroxypyridin-1(2H)-yl)-3-oxoprop-1-en-1-yl)-2-methoxybenzoate (Compound 9) (91 mg, 36.1%) as a white solid.

¹H NMR (400 MHz, Chloroform-d) δ 8.01 (d, J=2.3 Hz, 1H), 7.77-7.68 (m, 2H), 7.42 (d, J=15.6 Hz, 1H), 7.36 (t, J=4.6 Hz, 1H), 6.98 (d, J=8.7 Hz, 1H), 4.10 (t, J=6.5 Hz, 2H), 3.95 (s, 3H), 3.91 (s, 3H), 2.53 (td, J=6.5, 4.5 Hz, 2H).

### Preparation Example 10

### Synthesis of (E)-methyl-5-(3-(3-bromo-2-oxo-5,6-dihydropyridin-1(2H)-yl)-3-oxoprop-1-en-1-yl)-2-hydroxybenzoate (Compound 10)

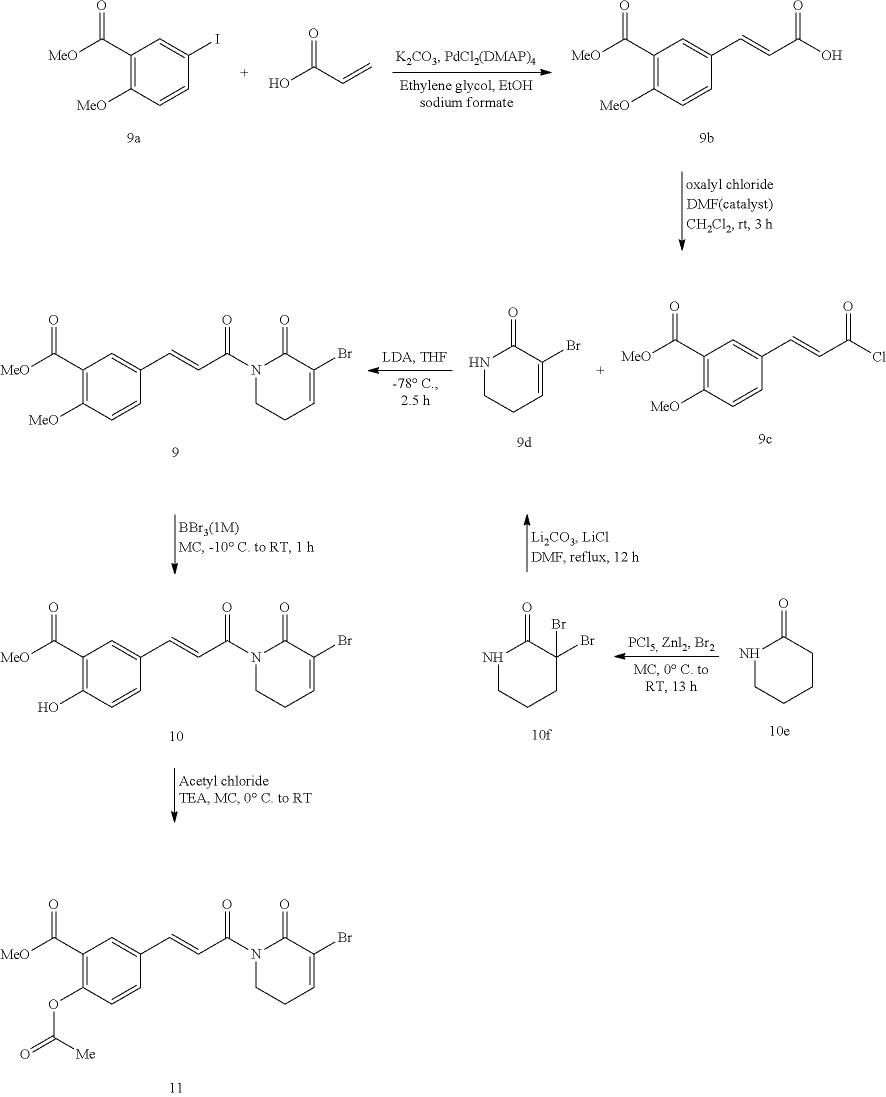

(E)-methyl-5-(3-(3-bromo-2-oxo-5,6-dihydropyridin-1(2H)-yl)-3-oxoprop-1-en-1-yl)-2-methoxybenzoate (Compound 9) (220 mg, 0.56 mmol) prepared in Preparation Example 9 and tetrahydrofuran (10.0 ml) were put into a round bottom flask under an argon atmosphere, the temperature was lowered to −10° C., and after 10 minutes, a tribromoboron solution (a 1.0 M methane dichloride solution, 1.60 ml, 1.67 mmol) was slowly added thereto, and the reaction temperature was allowed to gradually reach room temperature while stirring the mixture for 1 hour. It was confirmed by TLC whether the reaction was completed, and when the reaction was completed, the reaction temperature was again lowered to 0° C., and the reaction was terminated using 1.50 ml of a 1 N hydrochloric acid solution. After the mixture was stirred at the same temperature for another 10 minutes, the resulting product was extracted three times with methane dichloride, and the organic layer was washed once with a saturated sodium chloride solution. The organic layer was dried over anhydrous sodium sulfate, distilled under reduced pressure, and then separated by silica gel chromatography (ethyl acetate/hexane=1/3 to 1/2) to obtain (E)-methyl-5-(3-(3-bromo-2-oxo-5,6-dihydropyridin-1(2H)-yl)-3-oxoprop-1-en-1-yl)-2-hydroxybenzo ate (Compound 10) (76.5 mg, 35.9%) as a white solid.

¹H NMR (400 MHz, Chloroform-d) δ 11.03 (s, 1H), 8.07 (d, J=2.3 Hz, 1H), 7.76-7.68 (m, 2H), 7.41 (d, J=15.6 Hz, 1H), 7.36 (t, J=4.6 Hz, 1H), 7.00 (d, J=8.6 Hz, 1H), 4.10 (t, J=6.5 Hz, 2H), 3.99 (s, 3H), 2.53 (td, J=6.5, 4.6 Hz, 2H).

### Preparation Example 11

### Synthesis of (E)-methyl-2-acetoxy-5-(3-(3-bromo-2-oxo-5,6-dihydropyridin-1(2H)-yl)-3-oxoprop-1-en-1-yl)benzoate (Compound 11)

(E)-methyl-5-(3-(3-bromo-2-oxo-5,6-dihydropyridin-1(2H)-yl)-3-oxoprop-1-en-1-yl)-2-hydroxybenzoate (Compound 10) (30 mg, 0.08 mmol) prepared in Preparation Example 10 and methane dichloride (2.0 ml) were put into a round bottom flask under an argon atmosphere, and the temperature was lowered to 0° C. After 10 minutes, triethylamine (20.3 μl, 0.16 mmol) was added thereto, and the resulting mixture was stirred for 5 minutes. After acetate chloride (11.4, 0.16 mmol) was slowly added thereto at the same temperature, the resulting mixture was stirred for 1 hour while maintaining the temperature at 0° C. It was confirmed by TLC whether the reaction was completed, and when the reaction was completed, and the reaction was terminated using 2.0 μl of distilled water. Thereafter, extraction was performed three times with methane dichloride and the organic layer was washed once with a saturated sodium chloride solution. The organic layer was dried over anhydrous sodium sulfate, distilled under reduced pressure, and then separated by silica gel chromatography (ethyl acetate/hexane=1/3 to 1/2) to obtain (E)-methyl-2-acetoxy-5-(3-(3-bromo-2-oxo-5,6-dihydropyridin-1 (2H)-yl)-3-oxoprop-1-en-1-yl)benzoate (Compound 11) (34.0 mg, 87.4%) as a yellow solid.

¹H NMR (400 MHz, Chloroform-d) δ 8.20 (d, J=2.2 Hz, 1H), 7.78 (dd, J=8.5, 2.4 Hz, 1H), 7.74 (d, J=15.7 Hz, 1H), 7.49 (d, J=15.6 Hz, 1H), 7.37 (t, J=4.6 Hz, 1H), 7.13 (d, J=8.4 Hz, 1H), 4.11 (t, J=6.5 Hz, 2H), 3.90 (s, 3H), 2.54 (td, J=6.4, 4.5 Hz, 2H), 2.36 (s, 3H).

### Preparation Example 12

### Synthesis of (E)-methyl-5-(3-(3-chloro-2-oxo-5,6-dihydropyridin-1(2H)-yl)-3-oxoprop-1-en-1-yl)-2-hydroxybenzoate (Compound 12)

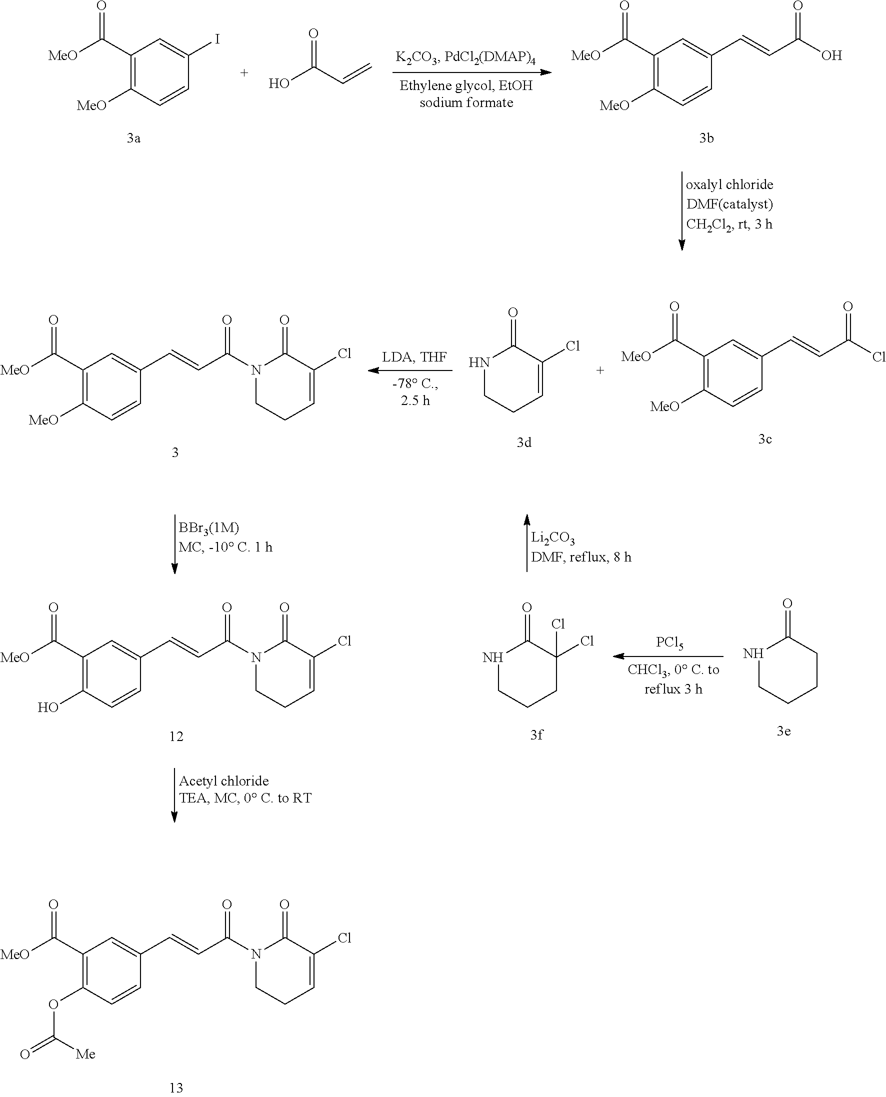

(E)-methyl-5-(3-(3-chloro-2-oxo-5,6-dihydroxypyridin-1(2H)-yl)-3-oxoprop-1-en-1-yl)-2-methoxybenzoate (Compound 3) (240 mg, 0.69 mmol) prepared in Preparation Example 3 and tetrahydrofuran (13.0 ml) were put into a round bottom flask under an argon atmosphere, and the temperature was lowered to −10° C. After 10 minutes, a tribromoboron solution (a 1.0 M dichloride methane solution, 2.06 ml, 2.06 mmol) was slowly added thereto, the resulting mixture was stirred for 1 hour, and the reaction temperature was allowed to gradually reach room temperature. It was confirmed by TLC whether the reaction was completed, and when the reaction was completed, the reaction temperature was again lowered to 0° C., and the reaction was terminated using 1.50 ml of a 1 N hydrochloric acid solution. After the mixture was stirred at the same temperature for another 10 minutes, the resulting product was extracted three times with methane dichloride, and the organic layer was washed once with a saturated sodium chloride solution. The organic layer was dried over anhydrous sodium sulfate, distilled under reduced pressure, and then separated by silica gel chromatography (ethyl acetate/hexane=1/3 to 1/2) to obtain (E)-methyl-5-(3-(3-chloro-2-oxo-5,6-dihydropyridin-1(2H)-yl)-3-oxoprop-1-en-1-yl)-2-hydroxybenzo ate (Compound 12) (55.7 mg, 24.1%) as a white solid.

¹H NMR (400 MHz, Chloroform-d) δ 11.03 (s, 1H), 8.08 (d, J=2.5 Hz, 1H), 7.76-7.69 (m, 2H), 7.42 (d, J=15.6 Hz, 1H), 7.10 (t, J=4.6 Hz, 1H), 7.00 (d, J=8.6 Hz, 1H), 4.09 (t, J=6.5 Hz, 2H), 3.99 (s, 3H), 2.57 (td, J=6.5, 4.6 Hz, 2H).

### Preparation Example 13

### Synthesis of (E)-methyl-2-acetoxy-5-(3-(3-chloro-2-oxo-5,6-dihydropyridin-1(2H)-yl)-3-oxoprop-1-en-1-yl)benzoate (Compound 13)

(E)-methyl-5-(3-(3-chloro-2-oxo-5,6-dihydropyridin-1(2H)-yl)-3-oxoprop-1-en-1-yl)-2-hydroxybenzoate (Compound 12) (41 mg, 0.12 mmol) prepared in Preparation Example 12 and methane dichloride (2.0 ml) were put into a round bottom flask under an argon atmosphere, and the temperature was lowered to 0° C. After 10 minutes, triethylamine (33.5 μl, 0.24 mmol) was added thereto, and the resulting mixture was stirred for 5 minutes. After acetate chloride (17.1 μl, 0.24 mmol) was slowly added thereto at the same temperature, the resulting mixture was stirred for 1 hour while maintaining the temperature at 0° C. It was confirmed by TLC whether the reaction was completed, and when the reaction was completed, the reaction was terminated using 2.0 ml of distilled water. Extraction was performed three times with methane dichloride and the organic layer was washed once with a saturated sodium chloride solution. The organic layer was dried over anhydrous sodium sulfate, distilled under reduced pressure, and then separated by silica gel chromatography (ethyl acetate/hexane=1/3 to 1/2) to obtain (E)-methyl-2-acetoxy-5-(3-(3-chloro-2-oxo-5,6-dihydropyridin-1(2H)-yl)-3-oxoprop-1-en-1-yl)benzoate (Compound 13) (30.0 mg, 66.2%) as a yellow solid.

¹H NMR (400 MHz, Chloroform-d) δ 8.20 (d, J=2.2 Hz, 1H), 7.81-7.71 (m, 2H), 7.50 (d, J=15.6 Hz, 1H), 7.15-7.09 (m, 2H), 4.10 (t, J=6.5 Hz, 2H), 3.90 (s, 3H), 2.59 (td, J=6.4, 4.6 Hz, 2H), 2.36 (s, 3H).

### Preparation Example 14

### Synthesis of (E)-1-(3-(3-nitrophenyl)acryloyl)-1H-pyrrol-2(5H)-one (Compound 14)

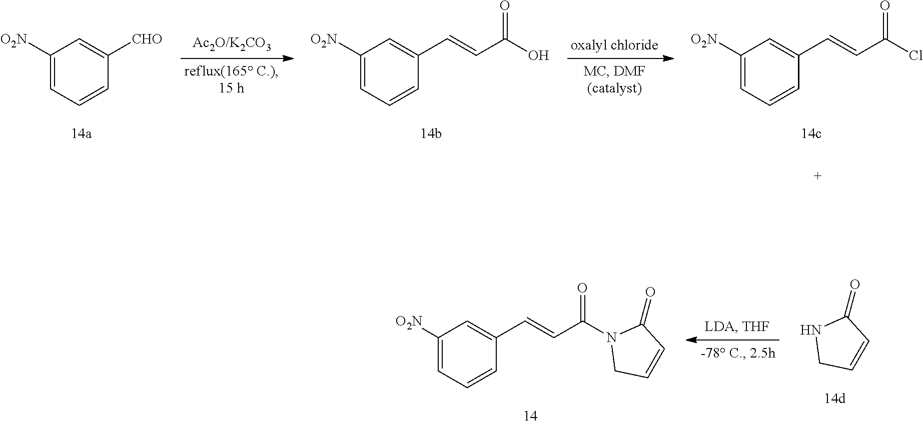 [SMILES: O=Cc1cccc([N+](=O)[O-])c1 ; O=C(O)/C=C/c1cccc([N+](=O)[O-])c1 ; O=C(Cl)/C=C/c1cccc([N+](=O)[O-])c1 ; O=C1C=CCN1C(=O)/C=C/c1cccc([N+](=O)[O-])c1 ; O=C1C=CCN1]

14-1. Preparation of (E)-3-(3-nitrophenyl)acrylic acid (Compound 14b)

Acetic anhydride (8.0 ml, 84.6 mmol) was slowly added to a round bottomed flask containing potassium carbonate (3.32 g, 24.0 mmol) under an argon atmosphere at 0° C., the resulting mixture was stirred for 5 minutes while gradually raising the temperature to room temperature, and then 3-nitrobenzalaldehyde (Compound 14a) (3.02 g, 20.0 mmol) was slowly added. The reaction mixture was stirred under reflux at 165° C. for 15 hours. When the reaction was completed, the temperature was lowered to room temperature, ice water was added thereto, and then the solid was filtered using a device, washed several times with water, and then dried. The resulting solid mixture was dissolved in an ethyl acetate (30.0 ml) solvent, washed twice with a saturated aqueous sodium bicarbonate solution (20.0 ml), and the ethyl acetate layer was discarded. The aqueous sodium bicarbonate solution layer was acidified with 3 N aqueous hydrogen chloride solution, and then extracted twice with an ethyl acetate solvent. The organic layer was washed once with a saturated sodium chloride solution, dried over anhydrous sodium sulfate, distilled under reduced pressure, and then sufficiently dried to obtain (E)-3-(3-nitrophenyl)acrylic acid (Compound 14b) (2.20 g, 56.8%) as a beige solid.

¹H NMR (400 MHz, CDCl₃+CD₃OD): δ 8.28 (1H, t, J=1.6 Hz), 8.14 (1H, ddd, J=8.4, 2.4, 1.2 Hz), 7.76 (1H, dt, J=8.0, 1.6 Hz), 7.62 (1H, d, J=16.0 Hz), 7.50 (1H, t, J=8.0 Hz), 6.46 (1H, d, J=16.0 Hz).

14-2. Preparation of (E)-1-(3-(3-nitrophenyl) acyloyl)-1H-pyrrol-2 (5H)-one (Compound 14) 1H-pyrrol-2(5H)-one (Compound 14d) (0.11 g, 1.25 mmol) was put into a 50-ml round bottom flask under an argon atmosphere, tetrahydrofuran (1.50 ml) was charged, then the temperature was lowered to −78° C., LDA (0.63 ml, 1.25 mmol, 2.0 M solution) was added thereto, and the resulting mixture was stirred for 45 minutes. (E)-3-(3-nitrophenyl)acryloyl chloride (Compound 14c) (0.22 g, 1.04 mmol) was dissolved in tetrahydrofuran (2.0 ml), the resulting solution was slowly added thereto at −78° C., and the resulting mixture was stirred for 1 hour. When the reaction was completed, the remaining LDA was decomposed with 1 N hydrochloric acid, and then extracted with ethyl acetate, and the ethyl acetate layer was washed with a saturated sodium chloride solution. The ethyl acetate layer was dried over anhydrous sodium sulfate, distilled under reduced pressure, and then separated by column chromatography (ethyl acetate/hexane=1/3 to 1/2) to obtain (E)-1-(3-(3-nitrophenyl)acryloyl)-1H-pyrrol-2(5H)-one (Compound 14) (81 mg, 30.2%) as a pale brown solid.

¹H NMR (400 MHz, Chloroform-d) δ 8.39 (t, J=2.0 Hz, 1H), 8.23 (ddd, J=8.2, 2.3, 1.1 Hz, 1H), 7.83 (dt, J=7.8, 1.4 Hz, 1H), 7.71 (d, J=16.0 Hz, 1H), 7.59 (t, J=7.9 Hz, 1H), 6.57 (d, J=16.0 Hz, 1H), 4.24 (t, J=6.7 Hz, 2H).

¹³C NMR (100 MHz, CDCl₃): δ 170.3, 164.6, 148.9, 147.4, 143.0, 136.8, 133.9, 130.1, 127.9, 124.8, 123.4, 121.9, 51.2.

### Preparation Example 15

### Synthesis of (E)-1-(3-(3-nitrophenyl)acryloyl)-5,6-dihydropyridin-2(1H)-one (Compound 15)

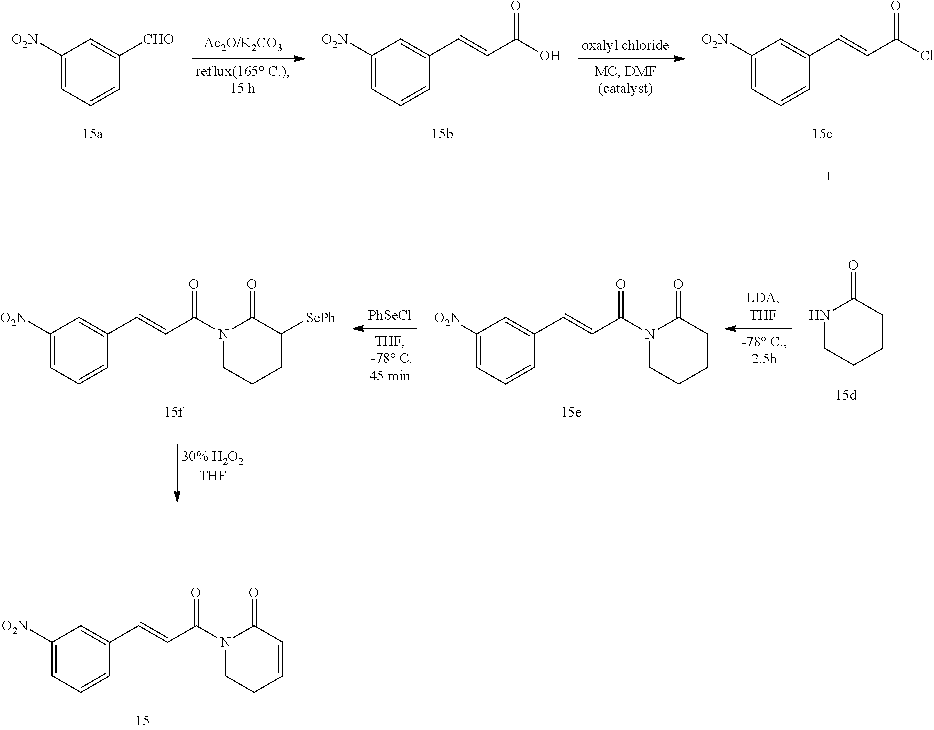

15-1. (E) -1-(3-(3 nitrophenyl)acryloyl)piperidin-2-one (Compound 15e)

2-piperidinone (Compound 15d) (0.14 g, 1.40 mmol) was put into a 25-ml round bottom flask under an argon atmosphere, tetrahydrofuran (1.50 ml) was charged, then the temperature was lowered to −78° C., LDA (0.70 ml, 1.40 mmol, 2.0 M solution) was added thereto, and the resulting mixture was stirred for 45 minutes. (E)-3-(3-nitrophenyl)acryloyl chloride (Compound 15c) (0.25 g, 1.16 mmol) was dissolved in tetrahydrofuran (1.80 ml), the resulting solution was slowly added thereto at −78° C., and the resulting mixture was stirred for 1 hour. When the reaction was completed, the remaining LDA was decomposed with 1 N hydrochloric acid, and then extracted twice with ethyl acetate, and the ethyl acetate layer was washed once with a saturated sodium chloride solution. The ethyl acetate layer was dried over anhydrous sodium sulfate, distilled under reduced pressure, and then separated by column chromatography (ethyl acetate/hexane=1/2 to 1/1) to obtain (E)-1-(3-(3-nitrophenyl)acryloyl)piperidin-2-one (Compound 15e) (165 mg, 52.1%) as a white solid.

¹H NMR (400 MHz, Chloroform-d) δ 8.39 (t, J=2.0 Hz, 1H), 8.20 (ddd, J=8.1, 2.2, 1.0 Hz, 1H), 7.86 (dt, J=7.8, 1.4 Hz, 1H), 7.67 (d, J=15.7 Hz, 1H), 7.56 (t, J=8.0 Hz, 1H), 7.50 (d, J=15.6 Hz, 1H), 3.82 (ddd, J=6.4, 4.2, 1.5 Hz, 2H), 2.67-2.59 (m, 2H), 1.95-1.86 (m, 4H).

15-2. Preparation of (E)-1-(3-(3-nitrophenyl)acryloyl)-3-(phenylselanyl)piperidin-2-one (Compound 15f)

(E)-1-(3-(3-nitrophenyl)acryloyl)piperidin-2-one (Compound 15e) (0.16 g, 0.60 mmol) prepared in Preparation Example 15-1 was put into a 25-ml round bottom flask under an argon atmosphere, tetrahydrofuran (3.50 ml) was charged, then the temperature was lowered to −78° C., LDA (0.40 ml, 0.78 mmol, 2.0 M solution) was added thereto, and the resulting mixture was stirred for 45 minutes. Phenylselenyl chloride (0.13 g, 0.66 mmol) was dissolved in tetrahydrofuran (3.50 ml) and slowly added at −78° C., and the resulting solution was stirred for 4 hours. When the reaction was completed, water was added to decompose the remaining LDA, and the mixture was further stirred at 0° C. for 15 minutes and extracted twice with methane dichloride. The organic layer was washed once with a saturated sodium chloride solution, dried over anhydrous sodium sulfate, distilled under reduced pressure, and then separated by silica gel chromatography (ethyl acetate/hexane=1/4 to 1/3) to obtain (E)-1-(3-(3-nitrophenyl)acryloyl)-3-(phenylselanyl)piperidin-2-one (Compound 15f) (30 mg, 11.7%) as a white solid.

¹H NMR (400 MHz, Chloroform-d) δ 8.33 (t, J=2.0 Hz, 1H), 8.21 (ddd, J=8.2, 2.3, 1.0 Hz, 1H), 7.78 (dt, J=7.8, 1.4 Hz, 1H), 7.72-7.69 (m, 2H), 7.65 (d, J=15.6 Hz, 1H), 7.56 (t, J=8.0 Hz, 1H), 7.43-7.34 (m, 3H), 7.10 (d, J=15.6 Hz, 1H), 4.13 (ddd, J=5.2, 4.2, 1.0 Hz, 1H), 4.08-3.95 (m, 1H), 3.70 (ddd, J=14.0, 9.7, 4.6 Hz, 1H), 2.46-2.28 (m, 1H), 2.25-2.05 (m, 2H), 1.90 (dt, J=14.3, 5.1 Hz, 1H).

15-3. Preparation of (E)-1-(3-(3-nitrophenyl)acryloyl)-5,6-dihydropyridin-2(1H)-one (Compound 15)

(E)-1-(3-(3-nitrophenyeacryloyl)-3-(phenylselanyl)piperidin-2-one (Compound 15f) (28 mg, 0.07 mmol) prepared in Preparation Example 15-2 was put into a 25-ml round bottom flask under an argon atmosphere, tetrahydrofuran (1.00 ml) was charged, then the temperature was lowered to 0° C., hydrogen peroxide (20 μl, 0.18 mmol, 30% solution) was added thereto, and the resulting mixture was stirred for 15 minutes, and then further stirred for another 30 minutes by raising the temperature to room temperature. When the reaction was completed, a saturated sodium bicarbonate solution was added and the mixture was extracted twice with methane dichloride. The organic layer was washed once with a saturated sodium chloride solution, dried over anhydrous sodium sulfate, distilled under reduced pressure, and then separated by silica gel chromatography (ethyl acetate/hexane=1/2) to obtain (E)-1-(3-(3-nitrophenyl)acryloyl)-5,6-dihydropyridin-2(1H)-one (Compound 15) (7.10 mg, 37.4%) as a white solid.

¹H NMR (400 MHz, Chloroform-d) δ 8.41 (t, J=2.0 Hz, 1H), 8.21 (ddd, J=8.1, 2.2, 1.0 Hz, 1H), 7.88 (d, J=7.7 Hz, 1H), 7.73 (d, J=15.7 Hz, 1H), 7.60-7.54 (m, 2H), 7.03-6.94 (m, 1H), 6.07 (dt, J=9.7, 1.9 Hz, 1H), 4.06 (t, J=6.5 Hz, 2H), 2.51 (tdd, J=6.4, 4.2, 1.9 Hz, 2H).

### Preparation Example 16

### Synthesis of (E)-3-chloro-1-(3-(3-nitrophenypacryloyl)-5,6-dihydropyridin-2(1H)-one (Compound 16)

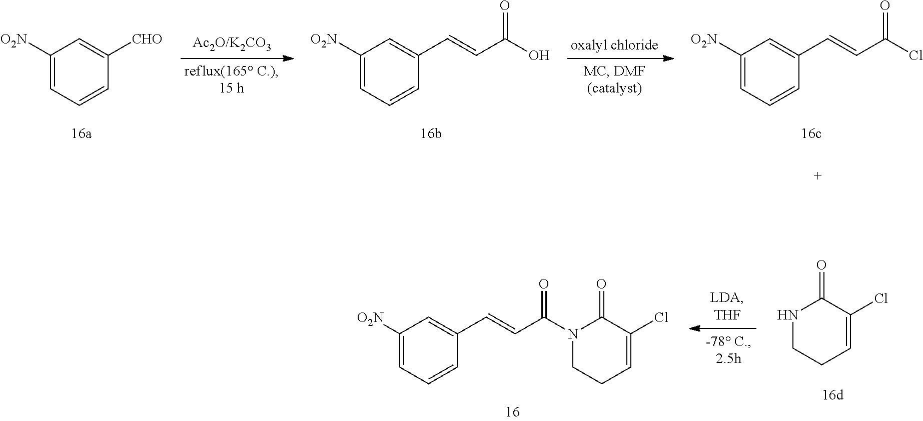 [SMILES: O=Cc1cccc([N+](=O)[O-])c1 ; O=C(O)/C=C/c1cccc([N+](=O)[O-])c1 ; O=C(Cl)/C=C/c1cccc([N+](=O)[O-])c1 ; O=C(/C=C/c1cccc([N+](=O)[O-])c1)N1CCC=C(Cl)C1=O ; O=C1NCCC=C1Cl]

3-chloro-5,6-dihydropyridin-2(1H)-one (Compound 16d) (0.17 g, 1.31 mmol) was put into a 25-ml round bottom flask under an argon atmosphere, tetrahydrofuran (2.0 ml) was charged, then the temperature was lowered to −78° C., LDA (0.70 ml, 1.31 mmol, 2.0 M solution) was added thereto, and the resulting mixture was stirred for 45 minutes. (E)-3-(3-nitrophenyl)acryloyl chloride (Compound 16c) (0.23 g, 1.09 mmol) was dissolved in tetrahydrofuran (1.50 ml), the resulting solution was slowly added thereto at −78° C., and the resulting mixture was stirred for 1 hour. When the reaction was completed, the remaining LDA was decomposed with 1 N hydrochloric acid, and extracted twice with ethyl acetate. The ethyl acetate layer was washed once with a saturated sodium chloride solution. The ethyl acetate layer was dried over anhydrous sodium sulfate, distilled under reduced pressure, and then separated by column chromatography (ethyl acetate/hexane=1/2 to 1/1) to obtain (E)-3-chloro-1-(3-(3-nitrophenyl)acryloyl)-5,6-dihydropyridin-2(1H)-one (Compound 16) (135 mg, 40.7%) as a pale yellow solid.

¹H NMR (400 MHz, Chloroform-d) δ 8.40 (t, J=2.0 Hz, 1H), 8.23 (ddd, J=8.2, 2.3, 1.0 Hz, 1H), 7.90 (d, J=7.7 Hz, 1H), 7.75 (d, J=15.7 Hz, 1H), 7.61-7.53 (m, 2H), 7.13 (t, J=4.6 Hz, 1H), 4.11 (t, J=6.5 Hz, 2H), 2.60 (td, J=6.5, 4.6 Hz, 2H).

### Preparation Example 17

### Synthesis of (E)-3-bromo-1-(3-(3-nitrophenypacryloyl)-5,6-dihydropyridin-2(1H)-one (Compound 17)

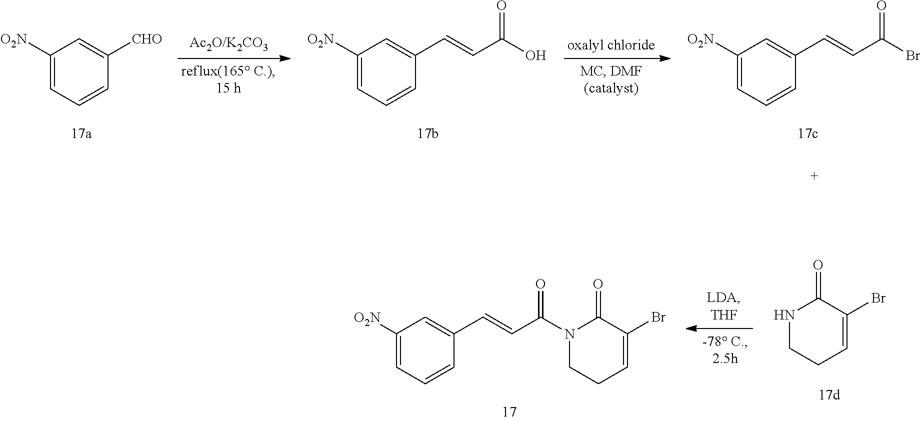 [SMILES: O=Cc1cccc([N+](=O)[O-])c1 ; O=C(O)/C=C/c1cccc([N+](=O)[O-])c1 ; O=C(Br)/C=C/c1cccc([N+](=O)[O-])c1 ; O=C(/C=C/c1cccc([N+](=O)[O-])c1)N1CCC=C(Br)C1=O ; O=C1NCCC=C1Br]

3-bromo-5,6-dihydropyridin-2(1H)-one (Compound 17d) (0.22 g, 1.25 mmol) was put into a 25-ml round bottom flask under an argon atmosphere, tetrahydrofuran (2.0 ml) was charged, then the temperature was lowered to −78° C., LDA (0.63 ml, 1.25 mmol, 2.0 M solution) was added thereto, and the resulting mixture was stirred for 45 minutes. (E)-3-(3-nitrophenyl)acryloyl chloride (Compound 17c) (0.22 g, 1.04 mmol) was dissolved in tetrahydrofuran (1.50 ml), the resulting solution was slowly added thereto at −78° C., and the resulting mixture was stirred for 1 hour. When the reaction was completed, the remaining LDA was decomposed with 1 N hydrochloric acid, and then extracted twice with ethyl acetate, and the ethyl acetate layer was washed once with a saturated sodium chloride solution. The ethyl acetate layer was dried over anhydrous sodium sulfate, distilled under reduced pressure, and then separated by column chromatography (ethyl acetate/hexane=1/2.5 to 1/2) to obtain (E)-3-bromo-1-(3-(3-nitrophenyl)acryloyl)-5,6-dihydropyridin-2(1H)-one (Compound 17) (110 mg, 30.1%) as a pale yellow solid.

¹H NMR (400 MHz, Chloroform-d) δ 8.40 (t, J=2.0 Hz, 1H), 8.22 (ddd, J=8.1, 2.3, 1.0 Hz, 1H), 7.90 (d, J=7.6 Hz, 1H), 7.75 (d, J=15.6 Hz, 1H), 7.62-7.53 (m, 2H), 7.39 (t, J=4.6 Hz, 1H), 4.12 (t, J=6.5 Hz, 2H), 2.56 (td, J=6.4, 4.6 Hz, 2H).

### Preparation Example 18

### Synthesis of (E)-1-(3-(2,3-dihydrobenzo[b][1,4]dioxin-6-yl)acryloyl)-1H-pyrrol-2(5H)-one (Compound 18)

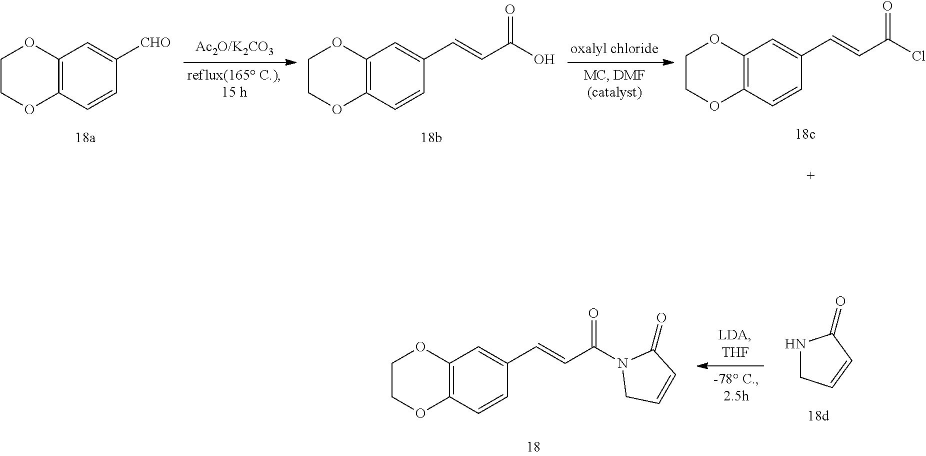 [SMILES: O=Cc1ccc2c(c1)OCCO2 ; O=C(O)/C=C/c1ccc2c(c1)OCCO2 ; O=C(Cl)/C=C/c1ccc2c(c1)OCCO2 ; O=C1C=CCN1C(=O)/C=C/c1ccc2c(c1)OCCO2 ; O=C1C=CCN1]

18-1. Preparation of (E)-3-(2,3-dihydrobenzo [b][1,4]dioxin-6-yl) acrylic acid (Compound 18b)

Acetic anhydride (4.0 ml, 42.2 mmol) was slowly added to a round bottom flask containing potassium carbonate (1.04 g, 7.54 mmol) under an argon atmosphere at 0° C., the temperature was gradually raised to room temperature, the mixture was stirred for 5 minutes, and then 2,3-dihydrobenzo[b][1,4]dioxine-6-carbaldehyde (Compound 18a) (0.99 g, 6.03 mmol) was slowly added thereto. The reaction mixture was stirred under reflux at 165° C. for 15 hours. When the reaction was completed, the temperature was lowered to room temperature (a solid was produced within 1 hour), ice water was added thereto, and then the solid was filtered using a device, washed several times with water, and then dried. The resulting solid mixture was dissolved in an ethyl acetate (30.0 ml) solvent, washed twice with a saturated aqueous sodium bicarbonate solution (20.0 ml), and the ethyl acetate layer was discarded. The aqueous sodium bicarbonate solution layer was acidified with 3 N aqueous hydrogen chloride solution, and then extracted twice with an ethyl acetate solvent. The organic layer was washed once with a saturated sodium chloride solution, dried over anhydrous magnesium sulfate, distilled under reduced pressure, and then sufficiently dried to obtain (E)-3-(2,3-dihydrobenzo[b][1,4]dioxin-6-yl)acrylic acid (Compound 18b) (0.48 g, 38.9%) as a white solid.

¹H NMR (400 MHz, CDCl₃): δ 7.67 (1H, d, J=16.0 Hz), 7.08 (1H, d, J=2.0 Hz), 7.06 (1H, dd, J=8.4, 2.0 Hz), 6.88 (1H, d, J=8.4 Hz), 6.29 (1H, d, J=15.6 Hz), 4.31-4.26 (4H, m).

18-2. Preparation of (E)-1-(3-(2,3-dihydrobenzo[b][1,4]dioxin-6-yl)acryloyl)-1H-pyrrol-2(5H)-one (Compound 18)

1H-pyrrol-2(5H)-one (Compound 18d) (58 mg, 0.69 mmol) was put into a 50-ml round bottom flask under an argon atmosphere, tetrahydrofuran (1.0 ml) was charged, then the temperature was lowered to −78° C., LDA (0.35 ml, 0.69 mmol, 2.0 M solution) was added thereto, and the resulting mixture was stirred for 45 minutes. (E)-3-(2,3-dihydrobenzo[b][1,4]dioxin-6-yl)acryloyl chloride (Compound 18c) (120 mg, 0.58 mmol) was dissolved in tetrahydrofuran (1.2 ml), the resulting solution was slowly added thereto at −78 C, and the resulting mixture stirred for 1 hour. When the reaction was completed, the remaining LDA was decomposed with 1 N hydrochloric acid, and then extracted with ethyl acetate, and the ethyl acetate layer was washed with a saturated sodium chloride solution. The ethyl acetate layer was dried over anhydrous magnesium sulfate, distilled under reduced pressure, and then separated by column chromatography (ethyl acetate/hexane=1/2 to 1/1) to obtain (E)-1-(3-(2,3-dihydrobenzo[b][1,4]dioxin-6-yl)acryloyl)-1H-pyrrol-2(5H)-one (Compound 18) (10 mg, 10.1%) as a white solid.

¹H NMR (400 MHz, Chloroform-d) δ 7.90 (d, J=15.7 Hz, 1H), 7.80 (d, J=15.7 Hz, 1H), 7.33 (dt, J=6.1, 2.0 Hz, 1H), 7.21-7.13 (m, 2H), 6.87 (d, J=8.3 Hz, 1H), 6.21 (dt, J=6.1, 1.9 Hz, 1H), 4.52 (t, J=2.0 Hz, 2H), 4.31-4.26 (m, 4H).

¹³C NMR (101 MHz, Chloroform-d) δ 170.21, 165.50, 146.74, 145.99, 145.90, 143.75, 128.68, 128.01, 122.72, 117.74, 117.44, 116.84, 64.68, 64.28, 51.18.

### Preparation Example 19

### Synthesis of (E)-3-chloro-1-(3-(4-((5-(3,5-dichlorophenyl)-1,3,4-oxadiazol-2-yl)methoxy)-3,5-dimethoxyphenyl)acryloyl)-5,6-dihydropyridin-2(1H)-one (Compound 19)

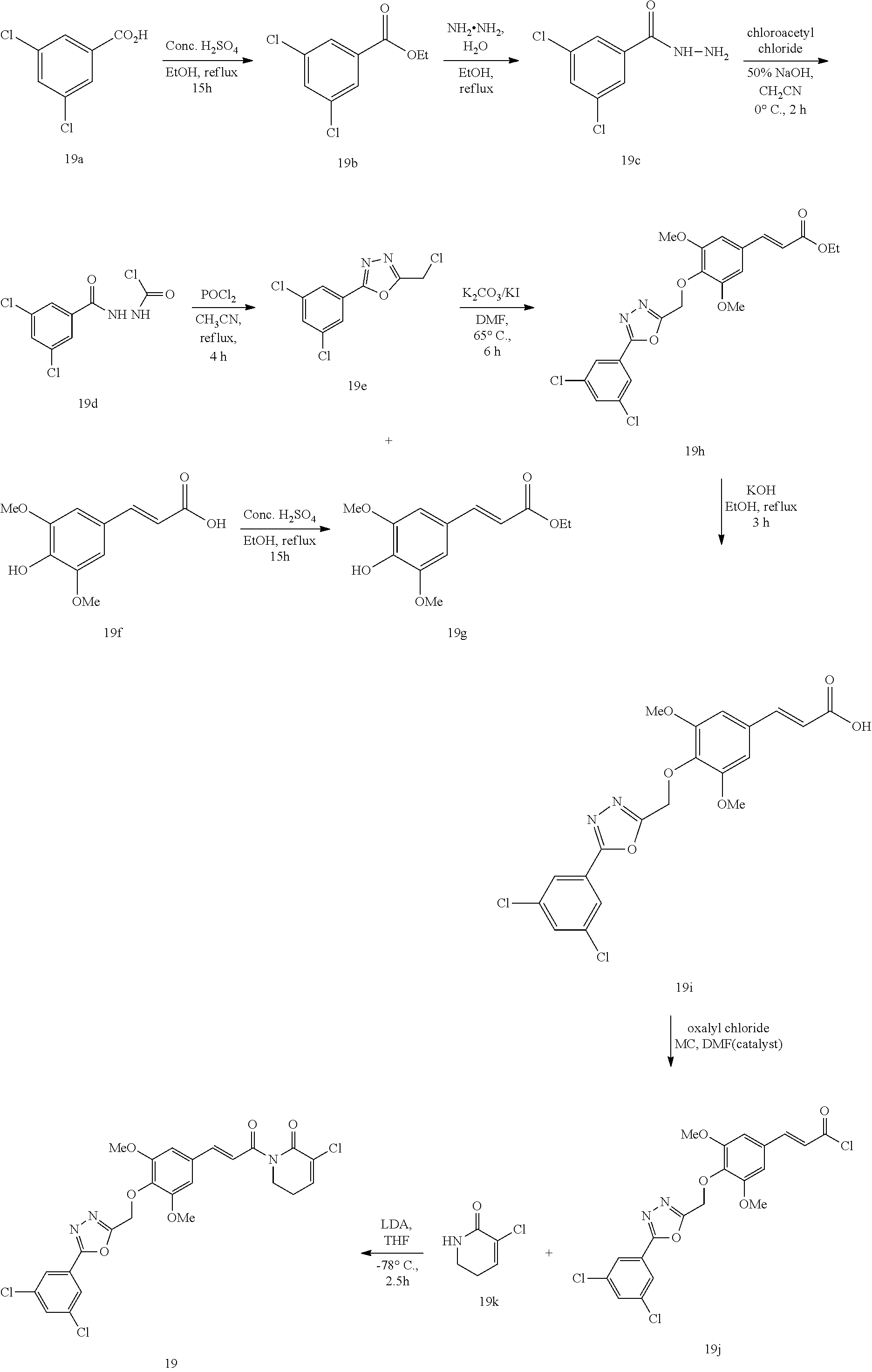

19-1. Preparation of ethyl-3,5-dichlorobenzoate (Compound 19b)

Concentrated sulfuric acid (0.29 ml) was slowly added to 3,5-dichlorobenzoic acid (Compound 19a) (1.05 g, 5.50 mmol) dissolved in an anhydrous ethanol (11.0 ml) solvent under an argon atmosphere at 0° C. The reaction mixture was stirred under reflux at 80° C. for 15 hours. When the reaction was completed, the temperature was lowered to room temperature, the solvent was evaporated under reduced pressure to remove all the solvent, and then the mixture was dissolved in an ethyl acetate (80.0 ml) solvent and washed twice with a saturated aqueous sodium bicarbonate solution (15.0 ml), twice with water (20.0 ml) and once with a saturated sodium chloride solution (20.0 ml). The mixture was dried over anhydrous magnesium sulfate, distilled under reduced pressure, and then sufficiently dried to obtain solid ethyl-3,5-dichlorobenzoate (Compound 19b) (1.15 g, 95.2%).

R_{f}=0.75 (ethyl acetate/hexane=1/5)

19-2. Preparation of 3,5-dichlorobenzohydrazide (Compound 19c)

Hydrazine hydrate (0.38 ml, 7.85 mmol) was put into ethyl-3,5-dichlorobenzoate (Compound 19b) (1.15 g, 5.24 mmol) dissolved in an anhydrous ethanol (12.0 ml) solvent under argon atmosphere at room temperature. The reaction mixture was stirred under reflux at 80° C. for 15 hours. When the reaction was completed, the temperature was lowered to room temperature, and the solvent was evaporated under reduced pressure and completely removed. After ice water was added to the mixture, the solid was filtered using a device, washed several times with water and hexane, and sufficiently dried to obtain 3,5-dichlorobenzohydrazide (Compound 19c) (0.98 g, 90.4%) as a white solid. The resulting product was used for the next reaction without further purification processes.

R_{f}=0.21 (ethyl acetate/hexane=1/1)

¹H NMR (400 MHz, DMSO-d₆): δ 10.00 (1H, br s), 7.83 (2H, d, J=1.6 Hz), 7.79 (1H, t, J=1.6 Hz), 4.59 (2H, br s).

19-3. Preparation of 2-(3,5-dichlorobenzoyl)hydrazinecarbonyl chloride) (Compound 19d)

Chloroacetyl chloride (0.45 ml, 5.68 mmol) was added to 3,5-dichlorobenzohydrazide (Compound 19c) (0.97 g, 4.73 mmol) dissolved in an anhydrous acetonitrile (18.0 ml) solvent under an argon atmosphere at 0° C., and then immediately, a 40% aqueous sodium hydroxide solution (1.5 eq) was slowly added thereto, and the resulting mixture was further stirred for 2 hours. When the reaction was completed, ice water (20.0 ml) was added thereto, and the solid was filtered using a filtering device, washed several times with water, and sufficiently dried. To dry the solid compound completely, the solid compound was dissolved in ethyl acetate (200.0 ml) solvent, dried over anhydrous magnesium sulfate and distilled under reduced pressure, and then 2-(3,5-dichlorobenzoyl) hydrazine carbonyl chloride (Compound 19d) (0.93 g, 70.0%) was obtained. The resulting product was used for the next reaction without further purification processes.

R_{f}=0.54 (ethyl acetate/hexane=1/1)

19-4. Preparation of 2-(chloromethyl)-5-(3,5-dichlorophenyl)-1,3,4-oxadiazole) (Compound 19e)

After phosphoryl chloride (0.62 ml, 6.63 mmol) was added to 2-(3,5-dichlorobenzoyl)hydrazine carbonyl chloride (Compound 19d) (0.93 g, 3.31 mmol) dissolved in an anhydrous acetonitrile (18.0 ml) solvent under an argon atmosphere at room temperature, the resulting mixture was stirred under reflux at 90° C. for 4 hours. When the reaction was completed, the temperature was lowered to room temperature, and the solvent was removed by distillation under reduced pressure. After a saturated aqueous sodium bicarbonate solution (15.0 ml) was put into the mixture, the resulting product was extracted three times with an ethyl acetate solvent (50.0 ml) and washed three times with water (30.0 ml) and once with a saturated sodium chloride solution (30 ml). The resulting product was dried over anhydrous magnesium sulfate, distilled under reduced pressure, and then separated by column chromatography (ethyl acetate/hexane=1/10) to obtain 2-(chloromethyl)-5-(3,5-dichlorophenyl)-1,3,4,-oxadiazole (Compound 19e) (0.68 g, 77.7%) as a white solid.

R_{f}=0.48 (ethyl acetate/hexane=1/5)

¹H NMR (400 MHz, CDCl₃): δ 7.98 (2H, d, J=2.0 Hz), 7.56 (1H, t, J=2.0 Hz), 4.79 (2H, s)

¹³C NMR (100 MHz, CDCl₃): δ 164.1, 163.0, 136.4, 132.3, 126.1, 125.6, 33.0.

19-5. Preparation of (E)-ethyl-3-(4-hydroxy-3,5-dimethoxyphenyl)acrylate) (Compound 19g)

Five drops of concentrated sulfuric acid were slowly added to (E)-3-(4-hydroxy-3,5-dimethoxyphenyl)acrylic acid (Compound 19f) (0.45 g, 2.0 mmol) dissolved in an anhydrous ethanol (10.0 ml) solvent under an argon atmosphere at 0° C. The reaction mixture was stirred under reflux at 80° C. for 15 hours. When the reaction was completed, the temperature was lowered to room temperature, the solvent was completely removed by distillation under reduced pressure, and then water (20.0 ml) was added to the mixture, and the resulting product was extracted three times with an ethyl acetate (35.0 ml) solvent. The organic layer was washed once with a saturated aqueous sodium bicarbonate solution (25.0 ml), three times with water (30.0 ml), and once with a saturated sodium chloride solution (30.0 ml). The organic layer was dried over anhydrous magnesium sulfate, distilled under reduced pressure, and then separated by column chromatography (ethyl acetate/hexane=1/10) to obtain (E)-ethyl-3-(4-hydroxy-3,5-dimethoxyphenyl)acrylate (Compound 19g) (0.47 g, 92.9%).

R_{f}=0.52 (ethyl acetate/hexane=2/3)

19-6. Preparation of (E)-ethyl-3-(4-((5-(3,5-dichlorophenyl)-1,3,4-oxadiazol-2-yl)methoxy)-3,5-dimethoxyphenyl)acrylate (Compound 19h)

Potassium carbonate was added to (E)-ethyl-3-(4-hydroxy-3,5-dimethoxyphenyl)acrylate (Compound 19g) (0.23 g, 0.91 mmol) dissolved in an anhydrous dimethylformamide (3.0 ml) solvent under an argon atmosphere, and the resulting mixture was stirred at room temperature for 5 minutes. 2-(chloromethyl)-5-(3,5-dichlorophenyl)-1,3,4,-oxadiazole (Compound 19e) (0.24 g, 0.91 mmol) and potassium iodide (0.02 g, 0.09 mmol) dissolved in an anhydrous dimethylformamide (3.0 ml) solvent were slowly added to the above mixture (Compound 19 g). The reaction mixture was stirred at 65° C. for 6 hours. When the reaction was completed, the temperature was lowered to room temperature, and the solid was filtered using a filtering device and washed with ethyl acetate (25.0 ml) and water (20.0 ml). Water (20.0 ml) was added to the filtered liquid and the resulting product was extracted twice with an ethyl acetate (50.0 ml) solvent. The organic layer was washed three times with water (25.0 ml) and once with a saturated sodium chloride solution (25.0 ml), dried over anhydrous magnesium sulfate, distilled under reduced pressure, and then separated by column chromatography (ethyl acetate/hexane=1/4 to 1/3) to obtain (E)-ethyl-3-(4-((5-(3,5-dichlorophenyl)-1,3,4-oxadiazol-2-yl)methoxy)-3,5-dimethoxyphenyl) acrylate (Compound 19h) (0.40 g, 91.8%) as a white fluffy solid.

R_{f}=0.45 (ethyl acetate/hexane=1/2)

¹H NMR (400 MHz, CDCl₃): δ 8.00 (2H, d, J=2.0 Hz), 7.58 (1H, d, J=16.0 Hz), 7.55 (1H, t, J=2.0 Hz), 6.73 (2H, s), 6.35 (1H, d, J=16.0 Hz), 5.29 (2H, s), 4.27 (2H, q, J=7.2 Hz), 1.34 (3H, t, J=7.2 Hz)

¹³C NMR (100 MHz, CDCl₃): δ 166.9, 163.7, 163.6, 153.6, 144.3, 137.0, 136.3, 132.0, 131.7, 126.4, 125.5, 118.5, 105.0, 63.7, 60.8, 56.3, 14.5.

19-7. Preparation of (E)-3-(4-((5-(3,5-dichlorophenyl)-1,3,4-oxadiazol-2-yl)methoxy)-3,5-dimethoxyphenyl)acrylic acid (Compound 19i)

Potassium hydroxide (0.12 g, 2.09 mmol) was added to (E)-ethyl-3-(4-((5-(3,5-dichlorophenyl)-1,3,4-oxadiazol-2-yl)methoxy)-3,5-dimethoxyphenyeacrylate (Compound 19h) (0.40 g, 0.83 mmol) dissolved in an anhydrous ethanol (7.0 ml) solvent under an argon atmosphere at room temperature, and the resulting mixture was stirred under reflux at 80° C. for 3 hours. When the reaction was completed, the temperature was lowered to room temperature, the solvent was completely removed by distillation under reduced pressure, then ice water (10.0 ml) was added to the mixture, the resulting mixture was acidified with 1 N hydrochloric acid, and then the solid was filtered using a filtering device. The solid compound was washed several times with water and sufficiently dried to obtain (E)-3-(4-((5-(3,5-dichlorophenyl)-1,3,4-oxadiazol-2-yl)methoxy)-3,5-dimethoxyphenyl)acrylic acid (Compound 19i) (0.29 g, 77.5%) as a yellow solid.

¹H NMR (400 MHz, DMSO-d₆): δ 10.10 (1H, br s), 7.90 (2H, d, J=2.0 Hz), 7.88 (1H, t, J=2.0 Hz), 7.53 (1H, d, J=16.0 Hz), 7.09 (2H, s), 6.58 (1H, d, J=16.0 Hz), 4.51 (2H, s), 3.86 (6H, s)

¹³C NMR (100 MHz, DMSO-d₆): δ 167.9, 167.0, 162.5, 152.4, 143.4, 137.3, 135.6, 134.4, 131.3, 130.6, 126.4, 6.9, 163.7, 163.6, 153.6, 144.3, 137.0, 136.3, 132.0, 131.7, 126.3, 119.6, 105.7, 70.8, 56.2.

19-8. Preparation of (E)-3-chloro-1-(3-(4-((5-(3,5-dichlorophenyl)-1,3,4-oxadiazol-2-yl)methoxy)-3,5-dimethoxyphenyl)acryloyl)-5,6-dihydropyridin-2(1H)-one (Compound 19)

3-chloro-5,6-dihydropyridin-2(1H)-one (Compound 19k) (25 mg, 0.19 mmol) was put into a 25-ml round bottom flask under an argon atmosphere, tetrahydrofuran (1.0 ml) was charged, then the temperature was lowered to −78° C., LDA (0.10 ml, 0.19 mmol, 2.0 M solution) was added thereto, and the resulting mixture was stirred for 45 minutes. (E)-3-(4-((5-(3,5-dichlorophenyl)-1,3,4-oxadiazol-2-yl)methoxy)-3,5-dimethoxyphenyl)acryl chloride (Compound 19j) (76 mg, 0.16 mmol) was dissolved in tetrahydrofuran (1.0 ml), the resulting solution was slowly added thereto at −78 C, and the resulting mixture stirred for 1 hour. When the reaction was completed, the remaining LDA was decomposed with 1 N hydrochloric acid, and then extracted twice with ethyl acetate, and the ethyl acetate layer was washed once with a saturated sodium chloride solution. The ethyl acetate layer was dried over anhydrous sodium sulfate, distilled under reduced pressure, and then separated by column chromatography (ethyl acetate/hexane=1/4 to 1/3) to obtain (E)-3-chloro-1-(3-(4-((5-(3,5-dichlorophenyl)-1,3,4-oxadiazol-2-yl)methoxy)-3,5dimethoxyphenyl)acryloyl)-5,6-dihydropyridin-2(1H)-one (Compound 19) (8.90 mg, 9.90%) as a pale brown solid.

¹H NMR (400 MHz, Chloroform-d) δ 8.00 (d, J=1.9 Hz, 2H), 7.69 (d, J=15.6 Hz, 1H), 7.55 (t, J=1.9 Hz, 1H), 7.44 (d, J=15.5 Hz, 1H), 7.10 (t, J=4.6 Hz, 1H), 6.68 (s, 2H), 5.28 (s, 2H), 4.10 (t, J=6.5 Hz, 2H), 3.81 (s, 6H), 2.58 (td, J=6.4, 4.5 Hz, 2H).

### Experimental Example 1

### Inhibitory Effect on Infection of Pseudovirus

The inhibitory effect of the piperlongumine-based compound prepared above on the infection of virus was confirmed using a pseudovirus. In this case, baculovirus, which expresses the novel coronavirus spike protein on the surface thereof, was used as the pseudovirus. Specifically, the extent to which a pseudovirus containing a fluorescent biosensor (Montana Molecular, Bozeman, USA) was introduced into host cells through the interaction between spike proteins on the surface of baculovirus and angiotensin-converting enzyme 2 (ACE2) present on the surface of host cells was confirmed through green fluorescence analysis.

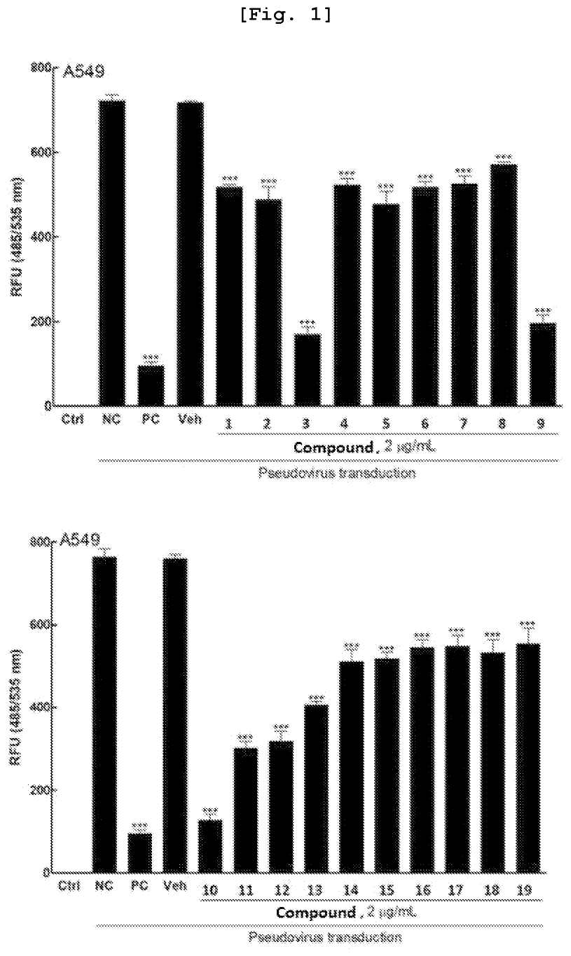

First, an A549 cell line, which is a human lung cancer cell line, was seeded in a 96-well plate at 3×10⁴ cells per well and cultured for 12 hours. The cultured cells were washed with a phosphate-buffered saline (PBS) buffer, and a DMEM medium containing 10% fetal bovine serum was added thereto. Meanwhile, a transduction mixture was prepared by mixing SARS-CoV-2 pseudovirus having a 3.3×10⁸ Vg/ml green fluorescent reporter with 2 mM sodium butyrate. Cells were treated with 2 μl/ml of each of Compounds 1 to 19 added to 50 μl of the transduction mixture. After the cells were cultured under 37° C. and 5% CO₂ conditions for 24 hours, the medium was removed, the same amount of PBS was added thereto, a Victor X2 Multilabel Microplate Reader (Perkin Elmer, USA) was used to measure fluorescence (excitation 485 nm and emission 535 nm). In this case, 50 μM chloroquine was used as a positive control. FIG. 1 illustrates the results of calculating the degree of infection with pseudovirus by a relative fluorescence unit (RFU) based on the value of the untreated control.

As illustrated in FIG. 1, the piperlongumine-based compound according to the present disclosure significantly suppressed pseudovirus from infecting host cells, confirming that compared to those treated with chloroquine, which is a positive control, at a concentration of 50 μM, the piperlongumine-based compound of the present disclosure exhibited an activity equal to or greater than that of the positive control. In particular, it could be seen that Compounds 3, 9, 10, 11, 12, and 13 exhibited an infection inhibitory activity of about 50% or more, and thus have better effects.

### Experimental Example 2

### Inhibitory Effect on Infection of Novel Coronavirus (SARS-CoV-2)

It was confirmed through a dose-response curve (DRC) experiment whether the piperlongumine-based compound prepared above inhibits the infection of the novel coronavirus.

First, a Vero cell line was seeded and cultured in 384-well tissue culture plates at 1.2×10⁴ cells per well. Meanwhile, Compound 1, 3, 4, 9, 10, 11, 12, 13, 16, 17 or 19 was prepared in dimethyl sulfoxide (DMSO) at 10 concentrations with the highest concentration of 50 μM. The cultured Vero cell line was treated with the compounds prepared above and further cultured for 1 hour. Thereafter, the cells were infected with the novel coronavirus at a multiplicity of infection (MOI) of 0.0125 and further cultured at 37° C. for 24 hours. Cultured cells were fixed with 4% paraformaldehyde and permeabilized by a typical method. Here, cells were stained by being treated with a primary antibody (Sino Biological) against the N protein of the novel coronavirus, and treated with an anti-rabbit IgG secondary antibody (Molecular Probes) conjugated with Alexaful 488 or Hoechst 33342 (Molecular Probes). Fluorescent images of the stained cells were acquired with Operetta (Perkin Elmer) and analyzed by Columbus software. In this case, the total number of cells per well was calculated by counting the number of Hoechst-stained nuclei, and the number of infected cells was calculated by counting the cells expressing a nucleocapsid (N) protein. In addition, the infection ratio was calculated as the number of cells expressing N protein/total number of cells in each well. The % inhibition of infection for each well was calculated by setting the average infection rate of wells containing cells (mock) not infected with coronavirus in the same plate to 100%, and normalizing the average infection rate of wells containing cells treated with 0.5% (v/v) DMSO instead of the compound to 0%. Response curves and IC₅₀ values according to the compound concentration were derived using XLFit4 (IDBS) software. Chloroquine (Sigma-aldrich), lopinavir (Selleck Chem) or remdesivir (MedChemExpress) were used as positive controls. As a result, calcutated IC₅₀ values are shown in the following Table 1.

TABLE 1

| | Infection inhibitory concentration IC₅₀ |
|---|---|
| Compound | (μM) |
| Chloroquine | 4.10 |
| Remdesivir | 2.65 |
| Lopinavir | 12.66 |
| Compound 1 | 4.46 |
| Compound 3 | 2.01 |
| Compound 4 | 7.68 |
| Compound 9 | 1.60 |
| Compound 10 | 2.31 |
| Compound 11 | 1.97 |
| Compound 12 | 2.63 |
| Compound 13 | 1.86 |
| Compound 16 | 4.19 |
| Compound 17 | 5.34 |
| Compound 19 | 4.12 |

As illustrated in Table 1, the piperlongumine-based compound according to the present disclosure significantly inhibited viral infection compared to lopinavir, which is used as a therapeutic agent for retroviral infections, and chloroquine, which is used as a therapeutic agent for novel coronavirus infections. In particular, Compounds 3, 9, 10, 11, 12 and 13 showed more remarkable effects than remdesivir, which is also used in severe patients with novel coronavirus infections.

Therefore, from the above results, it could be seen that the piperlongumine-based compound can be usefully used for the treatment of infections caused by RNA viruses such as novel coronavirus infections.

1. A pharmaceutical composition for preventing or treating viral infections, containing a compound represented by the following Chemical Formula 1, and a pharmaceutically acceptable salt or solvate thereof as an active ingredient: 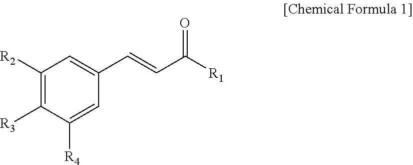
wherein, in Chemical Formula 1,
R₂ to R₄ are the same as or different from each other, and are each independently selected from the group consisting of hydrogen, deuterium, a halogen, a nitro group, a hydroxyl group, an amino group, a C₁˜C₄₀ alkyl group, a C₂˜C₄₀ alkenyl group, a C₂˜C₄₀ alkynyl group, a C₃˜C₄o cycloalkyl group, a heterocycloalkyl group having 3 to 40 nuclear atoms, a C₁˜C₂₀ alkoxy group, a C₁˜C₂₀ ketone group, a C₁˜C₂₀ ester group, a C₆˜C₂₀ aryl group, a heteroaryl group having 5 to 20 nuclear atoms, and a C₆˜C₂₀ aryloxy group, or these are optionally bonded to an adjacent group to form a C₆˜C₂₀ aryl or a heteroaryl ring having 5 to 20 nuclear atoms, provided that the case where R₂ to R₄ are the same is excluded,
R₁ is a substituent selected from the following structural formulae, 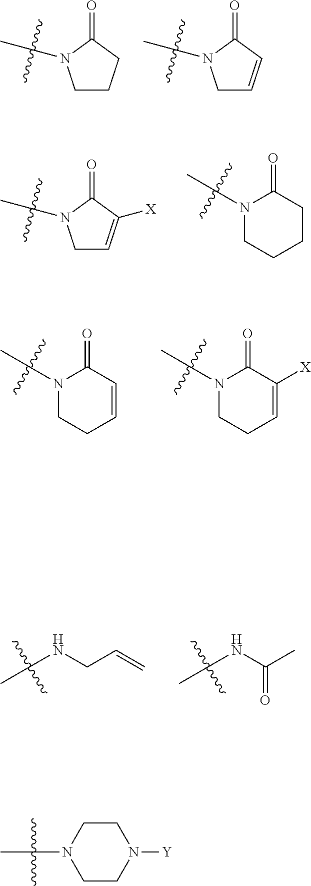
in the formulae,
the wavy part means a part that is bonded to Chemical Formula 1,
X is a halogen element selected from the group consisting of F, Cl, Br and I,
Y is a C₁˜C₁₀ alkyl group, and
the alkyl group, the ketone group, the ester group, the aryl group, and the heteroaryl group of R₂˜R₄ are optionally each independently substituted with one or more substituents selected from the group consisting of deuterium, a halogen, a cyano group, a nitro group, a C₁˜C₄₀ alkyl group, a C₂˜C₄₀ alkenyl group, a C₂˜C₄₀ alkynyl group, a C₆˜C₄₀ aryl group, a heteroaryl group having 5 to 40 nuclear atoms, a C₆˜C₄₀ aryloxy group, a C₁˜C₄₀ alkyloxy group, a C₆˜C₄₀ arylamine group, a C₃˜C₄₀ cycloalkyl group, a heterocycloalkyl group having 3 to 40 nuclear atoms, a C₁˜C₄₀ alkylsilyl group, a C₁˜C₄₀ alkylboron group, a C₆˜C₄₀ arylboron group, a C₆˜C₄₀ arylphosphine group, a C₆˜C₄₀ arylphosphine oxide group and a C₆˜C₄₀ arylsilyl group, and in this case, when there is a plurality of substituents, the substituents are optionally the same as or different from each other.

2. The pharmaceutical composition of claim 1, wherein R₁ has a substituent selected from the following structural formulae:

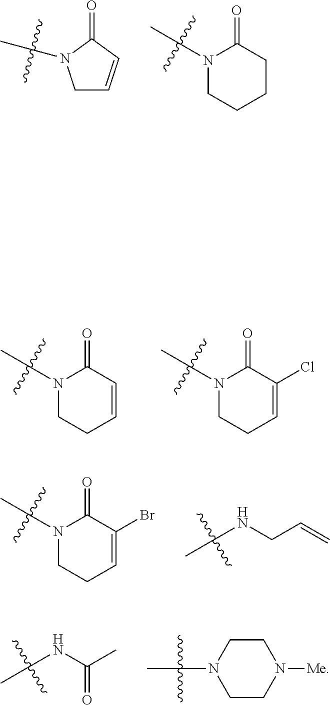

3. The pharmaceutical composition of claim 1, wherein the compound represented by Chemical Formula 1 is represented by any one of the following Chemical Formulae 2 to 5: 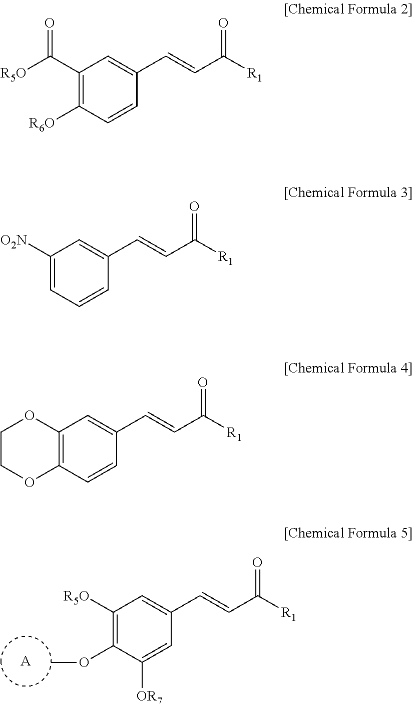
in Chemical Formulae 2 to 5,
R₁ is the same as that defined in claim 1,
R₅ to R₇ are the same as or different from each other, and are each independently selected from the group consisting of hydrogen, a hydroxyl group, a C₁˜C₆ alkyl group, a C₁˜C₆ alkoxy group, a C₁˜C₆ ketone group, and a C₁˜C₆ ester group,
Ring A is selected from the group consisting of a cycloalkyl ring, a heterocycloalkyl ring, an aryl ring and a heteroaryl ring, having 20 or less carbon atoms, and
the Ring A is optionally substituted with one or more substituents selected from the group consisting of deuterium, a halogen, a cyano group, a nitro group, a C₁˜C₄₀ alkyl group, a C₆˜C₄o aryl group, and a heteroaryl group having 5 to 40 nuclear atoms.

4. The pharmaceutical composition of claim 1, wherein the compound represented by Chemical Formula 1 is selected from the compound group represented by the following Chemical Formulae.

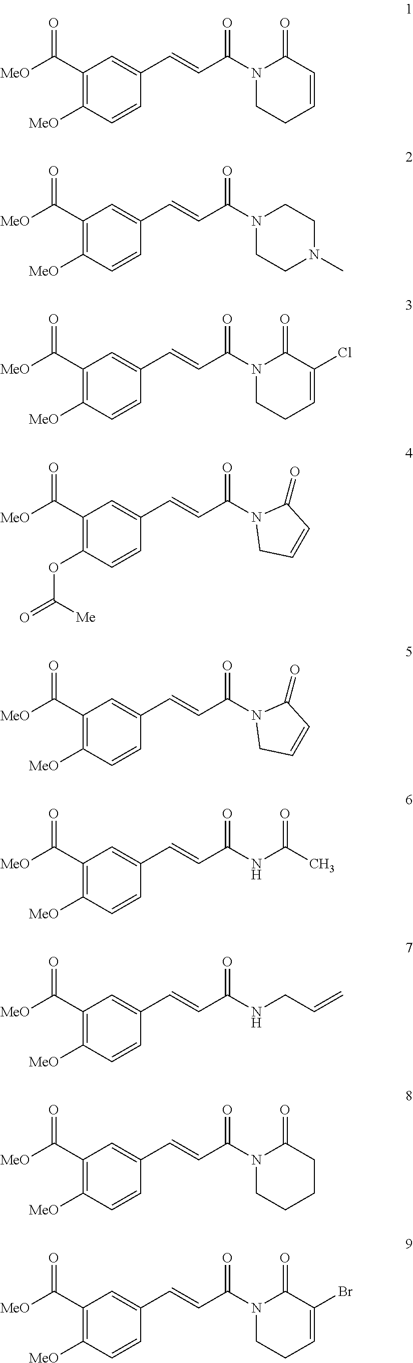

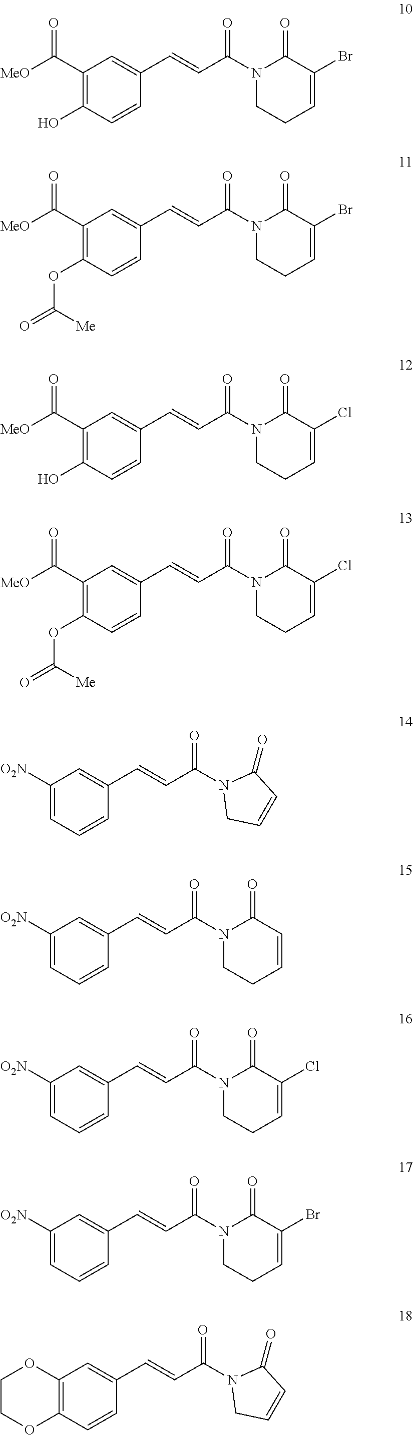

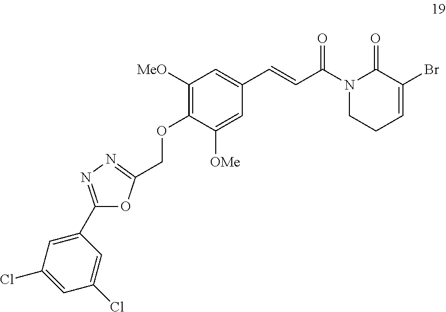 [SMILES: COc1cc(/C=C/C(=O)N2CCC=C(Br)C2=O)cc(OC)c1OCc1nnc(-c2cc(Cl)cc(Cl)c2)o1]

5. The pharmaceutical composition of claim 1, wherein the viral infections are infections by RNA virus.

6. The pharmaceutical composition of claim 5, wherein the RNA virus is a single-stranded positive RNA virus.

7. The pharmaceutical composition of claim 6, wherein the single-stranded positive RNA virus is a virus included in the family Coronaviridae, Picornaviridae, Flaviviridae, Togaviridae, or Caliciviridae.

8. The pharmaceutical composition of claim 6, wherein the single-stranded positive RNA virus is coronavirus, norovirus, dengue virus, rhinovirus, West Nile virus, Zika virus, hepatitis C virus, polio virus, or rubella virus.

9. The pharmaceutical composition of claim 8, wherein the coronavirus is novel coronavirus (SARS-CoV-2).

10. A health functional food for preventing or ameliorating viral infections, containing a compound represented by the following Chemical Formula 1, and a pharmaceutically acceptable salt or solvate thereof as an active ingredient: 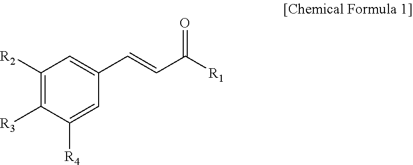
wherein, in Chemical Formula 1,
R₂ to R₄ are the same as or different from each other, and are each independently selected from the group consisting of hydrogen, deuterium, a halogen, a nitro group, a hydroxyl group, an amino group, a C₁˜C₄₀ alkyl group, a C₂˜C₄₀ alkenyl group, a C₂˜C₄₀ alkynyl group, a C₃˜C₄o cycloalkyl group, a heterocycloalkyl group having 3 to 40 nuclear atoms, a C₁˜C₂₀ alkoxy group, a C₁˜C₂₀ ketone group, a C₁˜C₂₀ ester group, a C₆˜C₂₀ aryl group, a heteroaryl group having 5 to 20 nuclear atoms, and a C₆˜C₂₀ aryloxy group, or these are optionally bonded to an adjacent group to form a C₆˜C₂₀ aryl or a heteroaryl ring having 5 to 20 nuclear atoms, provided that the case where R₂ to R₄ are the same is excluded,
R₁ is a substituent selected from the following structural formulae, 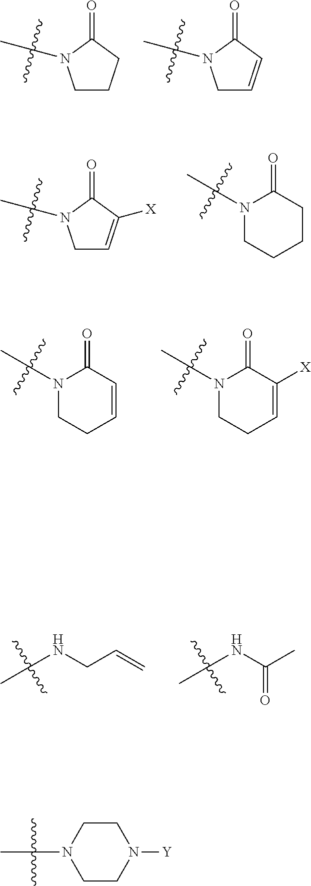
in the formulae,
the wavy part means a part that is bonded to Chemical Formula 1,
X is a halogen element selected from the group consisting of F, Cl, Br and I,
Y is a C₁˜C₁₀ alkyl group, and
the alkyl group, the ketone group, the ester group, the aryl group, and the heteroaryl group of R₂˜R₄ are optionally each independently substituted with one or more substituents selected from the group consisting of deuterium, a halogen, a cyano group, a nitro group, a C₁˜C₄₀ alkyl group, a C₂˜C₄₀ alkenyl group, a C₂˜C₄₀ alkynyl group, a C₆˜C₄₀ aryl group, a heteroaryl group having 5 to 40 nuclear atoms, a C₆˜C₄₀ aryloxy group, a C₁˜C₄₀ alkyloxy group, a C₆˜C₄₀ arylamine group, a C₃˜C₄₀ cycloalkyl group, a heterocycloalkyl group having 3 to 40 nuclear atoms, a C₁˜C₄₀ alkylsilyl group, a C₁˜C₄₀ alkylboron group, a C₆˜C₄₀ arylboron group, a C₆˜C₄₀ arylphosphine group, a C₆˜C₄₀ arylphosphine oxide group and a C₆˜C₄₀ arylsilyl group, and in this case, when there is a plurality of substituents, the substituents are optionally the same as or different from each other.

11. A method for preventing, ameliorating or treating viral infections, the method comprising administering the compound represented by Chemical Formula 1, and a pharmaceutically acceptable salt or solvate thereof to an individual: 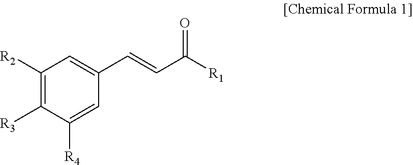
wherein, in Chemical Formula 1,
R₂ to R₄ are the same as or different from each other, and are each independently selected from the group consisting of hydrogen, deuterium, a halogen, a nitro group, a hydroxyl group, an amino group, a C₁˜C₄₀ alkyl group, a C₂˜C₄₀ alkenyl group, a C₂˜C₄₀ alkynyl group, a C₃˜C₄o cycloalkyl group, a heterocycloalkyl group having 3 to 40 nuclear atoms, a C₁˜C₂₀ alkoxy group, a C₁˜C₂₀ ketone group, a C₁˜C₂₀ ester group, a C₆˜C₂₀ aryl group, a heteroaryl group having 5 to 20 nuclear atoms, and a C₆˜C₂₀ aryloxy group, or these are optionally bonded to an adjacent group to form a C₆˜C₂₀ aryl or a heteroaryl ring having 5 to 20 nuclear atoms, provided that the case where R₂ to R₄ are the same is excluded,
R₁ is a substituent selected from the following structural formulae, 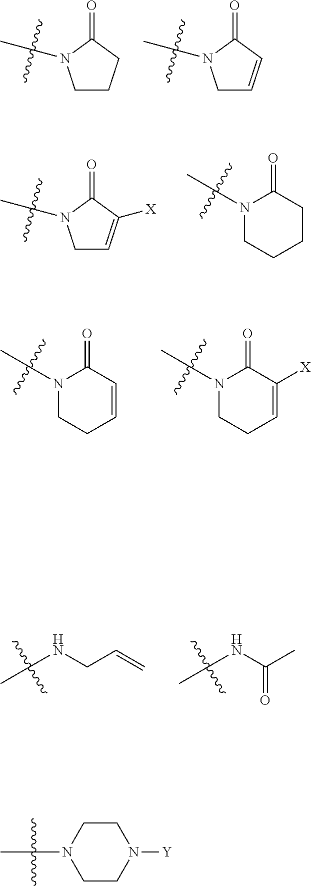
in the formulae,
the wavy part means a part that is bonded to Chemical Formula 1,
X is a halogen element selected from the group consisting of F, Cl, Br and I,
Y is a C₁˜C₁₀ alkyl group, and
the alkyl group, the ketone group, the ester group, the aryl group, and the heteroaryl group of R₂˜R₄ are optionally each independently substituted with one or more substituents selected from the group consisting of deuterium, a halogen, a cyano group, a nitro group, a C₁˜C₄₀ alkyl group, a C₂˜C₄₀ alkenyl group, a C₂˜C₄₀ alkynyl group, a C₆˜C₄₀ aryl group, a heteroaryl group having 5 to 40 nuclear atoms, a C₆˜C₄₀ aryloxy group, a C₁˜C₄₀ alkyloxy group, a C₆˜C₄₀ arylamine group, a C₃˜C₄₀ cycloalkyl group, a heterocycloalkyl group having 3 to 40 nuclear atoms, a C₁˜C₄₀ alkylsilyl group, a C₁˜C₄₀ alkylboron group, a C₆˜C₄₀ arylboron group, a C₆˜C₄₀ arylphosphine group, a C₆˜C₄₀ arylphosphine oxide group and a C₆˜C₄₀ arylsilyl group, and in this case, when there is a plurality of substituents, the substituents are optionally the same as or different from each other.

12. (canceled)
